SEQUENCE LISTING (118 sequences)

SEQ ID NO: 1 - protein, 268 aa, Homo sapiens
MSLSFLLLLFFSHLILSAWAHGEKRLAPKGQPGPAATDRNPRGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKKPPSPIKPKIPLSAPRKNTNSVKYRLKFRFG

SEQ ID NO: 2 - DNA, 5399 bp, Homo sapiens
ggggaagcttcgcaggcgtgcacggagcagtgagatcactggcgttataaatatcccggtgccagcgcggagatccgctcgggtggcctctctcttcccctctccccttctcttccccgaggctatgtccacccggtgcggcgaggcgggcagagccagaggcacgcagccgcacaggggctacagagcccagaatcagccctacaagatgcacttaggacccccgcggctggaagaatgagcttgtccttcctcctcctcctcttcttcagccacctgatcctcagcgcctgggctcacggggagaagcgtctcgcccccaaagggcaacccggacccgctgccactgataggaaccctagaggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataatattcctcttggccttgtgagaaaccattctttcccctcaggagtttctataggtgtcttcagagttctgaagaaaaattactggacacagcttcagctatacttacactgtattgaagtcacgtcatttgtttcaatgtgactgaaacaaaatgttttttgataggaaggaaactggaattctttgtactaatacagggagcacactccttcagttcagcaagacataaagccttttgctttatgcttgagggatatttagaactttgtattttcggaaagttaaataacagggactacgtatttttctgacttttacagattaacctgaaagaacatacatgatacatttttatttttggtttccaaagaatattttgatgcagataaaatattttgttaacttttgtttttttttgtttgttttcttaaaagtacctctgcattgagcatattttcttacttttattattttaattaatatgacataagcaatcattttatgctgtttatgaattataaatgtgtttatagctcatttgtaatatggaaatcttttacatttttcctattcactgcacttttttattgtttttatttctagccatacctcagataatatgtttagttttacattttaaaatgtttaaattctctttcacagcaccaaaggctcagcttggatttgtgtgtatgtgtatgtcaattcatgacattatgtggaatcctaaacctttggtggctgggatatgatgggttagaagcaaggagaaaatataaggactttttgatggaattaaatgtgggaggtaaggaaaaggatttagaggtaaaagtacactaagtttgcaacatttattgagatctaagtctgtcttgccttcatttctctttttatctcccccttgccctcattcttgaacagctggaggaatacattttattctgtccatgaagcatacactatgaaattcaagtgcttaaaaatacttctatgactctctgctatcccactgtatagatccacagggagcaaacacttagaaatgatagagaactgaaggagatcaatggtttaacagttatccatgccaagtcccattgtcagaaatattcttattactcagtcaaacactctttgagcttcccttcctaaaggtaaccaatccagtgaatagatgtgcccttttataaggaaacttctgatgtttattaaaaaaactggccttttgatagaggtaacttaatttgggaatttgttgtgttgaaatggcatttaatttcaacctaaatactgactgctggacataaatcacagaaaatttaacttaagaaaatttacaaaatttattctcaggtaatcattttaataaagttctgcaaaatacacgtttatcttacattcagaaatgtggcaaaaaaggcatagctaaaggctaaacatatggctttagtagtaacaaaagggttcatagaaacttcatggtttgcatttaaacatgtttaaagtgtacttataaactatttttttcttaaagcaaactatgatttattttggtgcacaaatacaaagtggaaacttaccaaaattgaactagctaccatataagcagattgctttaatttgatgggaaaatagtacacacatatatataacaaataatatattaaaaaacccatccatcaactaaaacattatatgtatacatcagtatagtgttttattataaagccaattatctgattaagcattctttccactgaatgcataatgtttaaatagcataaaatgaaatgctacaaaaattgaactaatttatactttaaagtatttctgggttaaatgaaacaatgaaattttttagtatgttcaactctcatccaaatggcatatgaccctgtttacacagcctaaagctaaaaatattactctagtttattctaatctattgttaagtattgtgcactgtataccaagttcttagggcacatgaaaaattttagctgccaaacaggaactagtaaacatatgttcctaataagtgaagggaaagataataatgatggtcaacaataagccacgtcaatgcataagttgtataggctaaatgttgcttgtaggctacattaaactcaaatgtaatagtttatcttatactcctggtttgatttgattagcatattaacgtgaaagtaggatagctactaaatatatattatgcaagtcaggaatcattaatttcaaaatttaaagccatgctaaaattaaaaagaaaatattaaattacacaattacacttgtctttactggccatacaaaatgattttttttttttttttgagacagagtcttgctctgtcaccaggctggagtgcagtggcatgatctcggctcactgcaacctccaactccctggtttaagggattctcctgcctcagcctcccaagtagctgggattacagactcatgccaccacgccagctaatttttgtatttttagtagagacggggtttcaccatgttggtcaggatggtctcaatcctggcctcttgatagtcctgacctcatgatctgcccacctcggcctccccaaagtgctgggattacaggtacaatgatgtataattaatgcttagtgaagcataaagttacctacatcaattaattaaatgaacttatgtacagaaaacatgtataaatataagtctatactaatgcttacaactttctaagagggttcttgcttatgtagctttttattattttaagtaactagaaccaccaaatatcaaataaaattatttggttatggttatgttcatctaaacacaacaataacttttatattaatatttaggagtctattttgtctataggtgacaaacatctccagactaacatgtcagttttatcaattatattatgtttaattatttaagatttctttatgtggaacatctatagagataaatagaaattttcaataagatgtagtaacactgtgatttatctttcaagagtctctcttcacttccttctaaagagactaatttgagagtacaggtgcatattaattttcttggttctttcagctgaattatattggtccagaagttcaaaatcatgtgacaataataagggatactgacagaagttatttccaagtttgtgtatatattataaaaattacatatataaaactaaggcttttatttctgttatttttaagcttttatttcttgtagctaaaaataaaacatcataaatctggtaggtaaatttcttattaaatcaatcttgaaatagaaaatgtaataactttcttaccattaacattttttacccttccatagaagggagggaataaatcatgacttatcccattttcaataacaaaacgaaactatggcactaaccaaaaacttgcattctggcataatttttacagttgcagagaattgtttctgggctcattaaaaaaagtagtattgcagacattgctgcaatgggaagcagacaataacttcttaaaggaattctacacctcctttaagatttacttaattgctacatctaaattctgataatttaaaatccattttaggtgataaaattttttaaaagttttgaaggaaacctctggataaatggacaaggcctaatttttttttgtagtcaatccaactgtactggccaatttttgaaataagattatatgattaggtattagcagagacaaagagttacctcctccatcttactctgccctatttgaaagtctcaggggagaaaagggaacaagatgctgatccaacctgagtggagtcaggtgaggcatctttacatctaagaatttttttttaaattttattattattatacttcaagttctagggtacatgtccacaatgcacatgtctgtcacacatgcacacatgtgccatgctggtgtgctgcacccaccaacctgtcatccagcattaggtatatctcctaatgctatccctcccctctccacccaccccacagcaggccccggtatgtgatgttccccttcgtgtgtccatgtgttcttattgttcaattcccacctatgagtgagaatatgtggtgtttggtttttggtccttgcaatagtttgctgagaatgatggtttccagcttcatccatgtccctacaaagaacatgaactcatcattttttatggctgcatagtattccatggtgtatatgtgccacattttcttaatccagtctatcattgttggacatttgggttggttccaagtctttgctattgtgaatagtgctgcaataaacatatgtgtgcatgtgtctttaaaaaaaaaaa

SEQ ID NO: 3 - protein, 123 aa, Homo sapiens
MSLSFLLLLFFSHLILSAWAHGEKRLAPKGQPGPAATDRNPRGSSSRQSSSSAMSSSSASSSPAASLGSQGSGLEQSSFQWSPSGRRTGSLYCRVGIGFHLQIYPDGKVNGSHEANMLSQVHR

SEQ ID NO: 4 - DNA, 5295 bp, Homo sapiens
ggggaagcttcgcaggcgtgcacggagcagtgagatcactggcgttataaatatcccggtgccagcgcggagatccgctcgggtggcctctctcttcccctctccccttctcttccccgaggctatgtccacccggtgcggcgaggcgggcagagccagaggcacgcagccgcacaggggctacagagcccagaatcagccctacaagatgcacttaggacccccgcggctggaagaatgagcttgtccttcctcctcctcctcttcttcagccacctgatcctcagcgcctgggctcacggggagaagcgtctcgcccccaaagggcaacccggacccgctgccactgataggaaccctagaggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataatattcctcttggccttgtgagaaaccattctttcccctcaggagtttctataggtgtcttcagagttctgaagaaaaattactggacacagcttcagctatacttacactgtattgaagtcacgtcatttgtttcaatgtgactgaaacaaaatgttttttgataggaaggaaactggaattctttgtactaatacagggagcacactccttcagttcagcaagacataaagccttttgctttatgcttgagggatatttagaactttgtattttcggaaagttaaataacagggactacgtatttttctgacttttacagattaacctgaaagaacatacatgatacatttttatttttggtttccaaagaatattttgatgcagataaaatattttgttaacttttgtttttttttgtttgttttcttaaaagtacctctgcattgagcatattttcttacttttattattttaattaatatgacataagcaatcattttatgctgtttatgaattataaatgtgtttatagctcatttgtaatatggaaatcttttacatttttcctattcactgcacttttttattgtttttatttctagccatacctcagataatatgtttagttttacattttaaaatgtttaaattctctttcacagcaccaaaggctcagcttggatttgtgtgtatgtgtatgtcaattcatgacattatgtggaatcctaaacctttggtggctgggatatgatgggttagaagcaaggagaaaatataaggactttttgatggaattaaatgtgggaggtaaggaaaaggatttagaggtaaaagtacactaagtttgcaacatttattgagatctaagtctgtcttgccttcatttctctttttatctcccccttgccctcattcttgaacagctggaggaatacattttattctgtccatgaagcatacactatgaaattcaagtgcttaaaaatacttctatgactctctgctatcccactgtatagatccacagggagcaaacacttagaaatgatagagaactgaaggagatcaatggtttaacagttatccatgccaagtcccattgtcagaaatattcttattactcagtcaaacactctttgagcttcccttcctaaaggtaaccaatccagtgaatagatgtgcccttttataaggaaacttctgatgtttattaaaaaaactggccttttgatagaggtaacttaatttgggaatttgttgtgttgaaatggcatttaatttcaacctaaatactgactgctggacataaatcacagaaaatttaacttaagaaaatttacaaaatttattctcaggtaatcattttaataaagttctgcaaaatacacgtttatcttacattcagaaatgtggcaaaaaaggcatagctaaaggctaaacatatggctttagtagtaacaaaagggttcatagaaacttcatggtttgcatttaaacatgtttaaagtgtacttataaactatttttttcttaaagcaaactatgatttattttggtgcacaaatacaaagtggaaacttaccaaaattgaactagctaccatataagcagattgctttaatttgatgggaaaatagtacacacatatatataacaaataatatattaaaaaacccatccatcaactaaaacattatatgtatacatcagtatagtgttttattataaagccaattatctgattaagcattctttccactgaatgcataatgtttaaatagcataaaatgaaatgctacaaaaattgaactaatttatactttaaagtatttctgggttaaatgaaacaatgaaattttttagtatgttcaactctcatccaaatggcatatgaccctgtttacacagcctaaagctaaaaatattactctagtttattctaatctattgttaagtattgtgcactgtataccaagttcttagggcacatgaaaaattttagctgccaaacaggaactagtaaacatatgttcctaataagtgaagggaaagataataatgatggtcaacaataagccacgtcaatgcataagttgtataggctaaatgttgcttgtaggctacattaaactcaaatgtaatagtttatcttatactcctggtttgatttgattagcatattaacgtgaaagtaggatagctactaaatatatattatgcaagtcaggaatcattaatttcaaaatttaaagccatgctaaaattaaaaagaaaatattaaattacacaattacacttgtctttactggccatacaaaatgattttttttttttttttgagacagagtcttgctctgtcaccaggctggagtgcagtggcatgatctcggctcactgcaacctccaactccctggtttaagggattctcctgcctcagcctcccaagtagctgggattacagactcatgccaccacgccagctaatttttgtatttttagtagagacggggtttcaccatgttggtcaggatggtctcaatcctggcctcttgatagtcctgacctcatgatctgcccacctcggcctccccaaagtgctgggattacaggtacaatgatgtataattaatgcttagtgaagcataaagttacctacatcaattaattaaatgaacttatgtacagaaaacatgtataaatataagtctatactaatgcttacaactttctaagagggttcttgcttatgtagctttttattattttaagtaactagaaccaccaaatatcaaataaaattatttggttatggttatgttcatctaaacacaacaataacttttatattaatatttaggagtctattttgtctataggtgacaaacatctccagactaacatgtcagttttatcaattatattatgtttaattatttaagatttctttatgtggaacatctatagagataaatagaaattttcaataagatgtagtaacactgtgatttatctttcaagagtctctcttcacttccttctaaagagactaatttgagagtacaggtgcatattaattttcttggttctttcagctgaattatattggtccagaagttcaaaatcatgtgacaataataagggatactgacagaagttatttccaagtttgtgtatatattataaaaattacatatataaaactaaggcttttatttctgttatttttaagcttttatttcttgtagctaaaaataaaacatcataaatctggtaggtaaatttcttattaaatcaatcttgaaatagaaaatgtaataactttcttaccattaacattttttacccttccatagaagggagggaataaatcatgacttatcccattttcaataacaaaacgaaactatggcactaaccaaaaacttgcattctggcataatttttacagttgcagagaattgtttctgggctcattaaaaaaagtagtattgcagacattgctgcaatgggaagcagacaataacttcttaaaggaattctacacctcctttaagatttacttaattgctacatctaaattctgataatttaaaatccattttaggtgataaaattttttaaaagttttgaaggaaacctctggataaatggacaaggcctaatttttttttgtagtcaatccaactgtactggccaatttttgaaataagattatatgattaggtattagcagagacaaagagttacctcctccatcttactctgccctatttgaaagtctcaggggagaaaagggaacaagatgctgatccaacctgagtggagtcaggtgaggcatctttacatctaagaatttttttttaaattttattattattatacttcaagttctagggtacatgtccacaatgcacatgtctgtcacacatgcacacatgtgccatgctggtgtgctgcacccaccaacctgtcatccagcattaggtatatctcctaatgctatccctcccctctccacccaccccacagcaggccccggtatgtgatgttccccttcgtgtgtccatgtgttcttattgttcaattcccacctatgagtgagaatatgtggtgtttggtttttggtccttgcaatagtttgctgagaatgatggtttccagcttcatccatgtccctacaaagaacatgaactcatcattttttatggctgcatagtattccatggtgtatatgtgccacattttcttaatccagtctatcattgttggacatttgggttggttccaagtctttgctattgtgaatagtgctgcaataaacatatgtgtgcatgtgtctttaaaaaaaaaaa

SEQ ID NO: 5 - DNA, 19 bp, Artificial Sequence
  Description of Artificial Sequence Synthetic oligonucleotide
ggtatgtggccctgaataa

SEQ ID NO: 6 - DNA, 19 bp, Artificial Sequence
  Description of Artificial Sequence Synthetic oligonucleotide
gcagagtgggcatcggttt

SEQ ID NO: 7 - DNA, 19 bp, Artificial Sequence
  Description of Artificial Sequence Synthetic oligonucleotide
ccacgaagccaatatgtta

SEQ ID NO: 8 - DNA, 19 bp, Artificial Sequence
  Description of Artificial Sequence Synthetic oligonucleotide
ataccaactcagtgaaata

SEQ ID NO: 9 - DNA, 19 bp, Artificial Sequence
  Description of Artificial Sequence Synthetic oligonucleotide
cctgaataaaagaggaaaa

SEQ ID NO: 10 - DNA, 19 bp, Artificial Sequence
  Description of Artificial Sequence Synthetic oligonucleotide
ggaaatatttgctgtgtct

SEQ ID NO: 11 - DNA, 19 bp, Artificial Sequence
  Description of Artificial Sequence Synthetic oligonucleotide
cagcatatctctacccatt

SEQ ID NO: 12 - DNA, 19 bp, Artificial Sequence
  Description of Artificial Sequence Synthetic oligonucleotide
taaaagaggaaaagccaaa

SEQ ID NO: 13 - DNA, 19 bp, Artificial Sequence
  Description of Artificial Sequence Synthetic oligonucleotide
caagtttcgctttggataa

SEQ ID NO: 14 - DNA, 19 bp, Artificial Sequence
  Description of Artificial Sequence Synthetic oligonucleotide
cagagcagcagtagcgcta

SEQ ID NO: 15 - DNA, 19 bp, Artificial Sequence
  Description of Artificial Sequence Synthetic oligonucleotide
agatctacccggatggcaa

SEQ ID NO: 16 - DNA, 19 bp, Artificial Sequence
  Description of Artificial Sequence Synthetic oligonucleotide
gcaatacatagaactgaaa

SEQ ID NO: 17 - DNA, 19 bp, Artificial Sequence
  Description of Artificial Sequence Synthetic oligonucleotide
tgtaggaatacgaggagtt

SEQ ID NO: 18 - DNA, 19 bp, Artificial Sequence
  Description of Artificial Sequence Synthetic oligonucleotide
gcaagttcagggagcgttt

SEQ ID NO: 19 - DNA, 19 bp, Artificial Sequence
  Description of Artificial Sequence Synthetic oligonucleotide
gtgccaagttcacagatga

SEQ ID NO: 20 - DNA, 19 bp, Artificial Sequence
  Description of Artificial Sequence Synthetic oligonucleotide
agcaatacatagaactgaa

SEQ ID NO: 21 - DNA, 19 bp, Artificial Sequence
  Description of Artificial Sequence Synthetic oligonucleotide
tcaggggattgtaggaata

SEQ ID NO: 22 - DNA, 19 bp, Artificial Sequence
  Description of Artificial Sequence Synthetic oligonucleotide
acagatgactgcaagttca

SEQ ID NO: 23 - DNA, 19 bp, Artificial Sequence
  Description of Artificial Sequence Synthetic oligonucleotide
ctataatacctatgcctca

SEQ ID NO: 24 - DNA, 19 bp, Artificial Sequence
  Description of Artificial Sequence Synthetic oligonucleotide
aaataccaactcagtgaaa

SEQ ID NO: 25 - DNA, 19 bp, Artificial Sequence
  Description of Artificial Sequence Synthetic oligonucleotide
tcagtgaaatacagactca

SEQ ID NO: 26 - DNA, 19 bp, Artificial Sequence
  Description of Artificial Sequence Synthetic oligonucleotide
ggaagtggcttggagcaga

SEQ ID NO: 27 - DNA, 19 bp, Artificial Sequence
  Description of Artificial Sequence Synthetic oligonucleotide
gatctacccggatggcaaa

SEQ ID NO: 28 - DNA, 19 bp, Artificial Sequence
  Description of Artificial Sequence Synthetic oligonucleotide
gattgtaggaatacgagga

SEQ ID NO: 29 - DNA, 19 bp, Artificial Sequence
  Description of Artificial Sequence Synthetic oligonucleotide
tacctatgcctcagcaata

SEQ ID NO: 30 - DNA, 19 bp, Artificial Sequence
  Description of Artificial Sequence Synthetic oligonucleotide
gaaatacagactcaagttt

SEQ ID NO: 31 - DNA, 19 bp, Artificial Sequence
  Description of Artificial Sequence Synthetic oligonucleotide
tcaagtttcgctttggata

SEQ ID NO: 32 - DNA, 19 bp, Artificial Sequence
  Description of Artificial Sequence Synthetic oligonucleotide
gagcagagcagtttccagt

SEQ ID NO: 33 - DNA, 19 bp, Artificial Sequence
  Description of Artificial Sequence Synthetic oligonucleotide
aaatatttgctgtgtctca

SEQ ID NO: 34 - DNA, 19 bp, Artificial Sequence
  Description of Artificial Sequence Synthetic oligonucleotide
tggcaaagtcaatggatcc

SEQ ID NO: 35 - DNA, 19 bp, Artificial Sequence
  Description of Artificial Sequence Synthetic oligonucleotide
agtcaatggatcccacgaa

SEQ ID NO: 36 - DNA, 19 bp, Artificial Sequence
  Description of Artificial Sequence Synthetic oligonucleotide
cacgaagccaatatgttaa

SEQ ID NO: 37 - DNA, 19 bp, Artificial Sequence
  Description of Artificial Sequence Synthetic oligonucleotide
aagccaatatgttaagtgt

SEQ ID NO: 38 - DNA, 19 bp, Artificial Sequence
  Description of Artificial Sequence Synthetic oligonucleotide
gtatgtggccctgaataaa

SEQ ID NO: 39 - DNA, 19 bp, Artificial Sequence
  Description of Artificial Sequence Synthetic oligonucleotide
gccctgaataaaagaggaa

SEQ ID NO: 40 - DNA, 19 bp, Artificial Sequence
  Description of Artificial Sequence Synthetic oligonucleotide
tgaaatacagactcaagtt

SEQ ID NO: 41 - DNA, 19 bp, Artificial Sequence
  Description of Artificial Sequence Synthetic oligonucleotide
aggaaaactccatgcaagt

SEQ ID NO: 42 - DNA, 19 bp, Artificial Sequence
  Description of Artificial Sequence Synthetic oligonucleotide
tggtatgtggccctgaata

SEQ ID NO: 43 - DNA, 19 bp, Artificial Sequence
  Description of Artificial Sequence Synthetic oligonucleotide
ggcttggagcagagcagtt

SEQ ID NO: 44 - DNA, 19 bp, Artificial Sequence
  Description of Artificial Sequence Synthetic oligonucleotide
gtaggaatacgaggagttt

SEQ ID NO: 45 - DNA, 19 bp, Artificial Sequence
  Description of Artificial Sequence Synthetic oligonucleotide
cctcagcaatacatagaac

SEQ ID NO: 46 - DNA, 19 bp, Artificial Sequence
  Description of Artificial Sequence Synthetic oligonucleotide
aataaaagaggaaaagcca

SEQ ID NO: 47 - DNA, 19 bp, Artificial Sequence
  Description of Artificial Sequence Synthetic oligonucleotide
ataaaagaggaaaagccaa

SEQ ID NO: 48 - DNA, 19 bp, Artificial Sequence
  Description of Artificial Sequence Synthetic oligonucleotide
actcagtgaaatacagact

SEQ ID NO: 49 - DNA, 19 bp, Artificial Sequence
  Description of Artificial Sequence Synthetic oligonucleotide
gagcagcagtagcgctatg

SEQ ID NO: 50 - DNA, 19 bp, Artificial Sequence
  Description of Artificial Sequence Synthetic oligonucleotide
atggcaaagtcaatggatc

SEQ ID NO: 51 - DNA, 19 bp, Artificial Sequence
  Description of Artificial Sequence Synthetic oligonucleotide
gggagtggtatgtggccct

SEQ ID NO: 52 - DNA, 19 bp, Artificial Sequence
  Description of Artificial Sequence Synthetic oligonucleotide
ccgctgccactgataggaa

SEQ ID NO: 53 - DNA, 19 bp, Artificial Sequence
  Description of Artificial Sequence Synthetic oligonucleotide
cagcagtagcgctatgtct

SEQ ID NO: 54 - DNA, 19 bp, Artificial Sequence
  Description of Artificial Sequence Synthetic oligonucleotide
aaggaagtggcttggagca

SEQ ID NO: 55 - DNA, 19 bp, Artificial Sequence
  Description of Artificial Sequence Synthetic oligonucleotide
ccacgaagccagtgtgtta

SEQ ID NO: 56 - DNA, 19 bp, Artificial Sequence
  Description of Artificial Sequence Synthetic oligonucleotide
agggagagattccaagaaa

SEQ ID NO: 57 - DNA, 19 bp, Artificial Sequence
  Description of Artificial Sequence Synthetic oligonucleotide
cctgaacaagagagggaaa

SEQ ID NO: 58 - DNA, 19 bp, Artificial Sequence
  Description of Artificial Sequence Synthetic oligonucleotide
ccacagaactgaaaagaca

SEQ ID NO: 59 - DNA, 19 bp, Artificial Sequence
  Description of Artificial Sequence Synthetic oligonucleotide
ggaaatatttgctgtgtct

SEQ ID NO: 60 - DNA, 19 bp, Artificial Sequence
  Description of Artificial Sequence Synthetic oligonucleotide
gactgtaagttcagggaga

SEQ ID NO: 61 - DNA, 19 bp, Artificial Sequence
  Description of Artificial Sequence Synthetic oligonucleotide
gggagagattccaagaaaa

SEQ ID NO: 62 - DNA, 19 bp, Artificial Sequence
  Description of Artificial Sequence Synthetic oligonucleotide
ggtacgtggccctgaacaa

SEQ ID NO: 63 - DNA, 19 bp, Artificial Sequence
  Description of Artificial Sequence Synthetic oligonucleotide
gtgccaaatttacggatga

SEQ ID NO: 64 - DNA, 19 bp, Artificial Sequence
  Description of Artificial Sequence Synthetic oligonucleotide
gtaagttcagggagagatt

SEQ ID NO: 65 - DNA, 19 bp, Artificial Sequence
  Description of Artificial Sequence Synthetic oligonucleotide
agatctacccggatggcaa

SEQ ID NO: 66 - DNA, 19 bp, Artificial Sequence
  Description of Artificial Sequence Synthetic oligonucleotide
agacaggccgagagtggta

SEQ ID NO: 67 - DNA, 19 bp, Artificial Sequence
  Description of Artificial Sequence Synthetic oligonucleotide
tgtaggaatacgaggagtt

SEQ ID NO: 68 - DNA, 19 bp, Artificial Sequence
  Description of Artificial Sequence Synthetic oligonucleotide
ccaaggaagcggctcggaa

SEQ ID NO: 69 - DNA, 19 bp, Artificial Sequence
  Description of Artificial Sequence Synthetic oligonucleotide
gaacatagcagtttccagt

SEQ ID NO: 70 - DNA, 19 bp, Artificial Sequence
  Description of Artificial Sequence Synthetic oligonucleotide
gcagagtgggcatcggttt

SEQ ID NO: 71 - DNA, 19 bp, Artificial Sequence
  Description of Artificial Sequence Synthetic oligonucleotide
tcaggggattgtaggaata

SEQ ID NO: 72 - DNA, 19 bp, Artificial Sequence
  Description of Artificial Sequence Synthetic oligonucleotide
acggatgactgtaagttca

SEQ ID NO: 73 - DNA, 19 bp, Artificial Sequence
  Description of Artificial Sequence Synthetic oligonucleotide
gttcaagcagtccgagcaa

SEQ ID NO: 74 - DNA, 19 bp, Artificial Sequence
  Description of Artificial Sequence Synthetic oligonucleotide
gaagtttcgctttggatga

SEQ ID NO: 75 - DNA, 19 bp, Artificial Sequence
  Description of Artificial Sequence Synthetic oligonucleotide
ggaagcggctcggaacata

SEQ ID NO: 76 - DNA, 19 bp, Artificial Sequence
  Description of Artificial Sequence Synthetic oligonucleotide
aagccagtgtgttaagtat

SEQ ID NO: 77 - DNA, 19 bp, Artificial Sequence
  Description of Artificial Sequence Synthetic oligonucleotide
catgcaagtgccaaattta

SEQ ID NO: 78 - DNA, 19 bp, Artificial Sequence
  Description of Artificial Sequence Synthetic oligonucleotide
gatctacccggatggcaaa

SEQ ID NO: 79 - DNA, 19 bp, Artificial Sequence
  Description of Artificial Sequence Synthetic oligonucleotide
gattgtaggaatacgagga

SEQ ID NO: 80 - DNA, 19 bp, Artificial Sequence
  Description of Artificial Sequence Synthetic oligonucleotide
ccatgcaagtgccaaattt

SEQ ID NO: 81 - DNA, 19 bp, Artificial Sequence
  Description of Artificial Sequence Synthetic oligonucleotide
gaactttccttcaccgtca

SEQ ID NO: 82 - DNA, 19 bp, Artificial Sequence
  Description of Artificial Sequence Synthetic oligonucleotide
gccagtgtgttaagtattt

SEQ ID NO: 83 - DNA, 19 bp, Artificial Sequence
  Description of Artificial Sequence Synthetic oligonucleotide
acaagagagggaaagccaa

SEQ ID NO: 84 - DNA, 19 bp, Artificial Sequence
  Description of Artificial Sequence Synthetic oligonucleotide
cctaggaacccgggagact

SEQ ID NO: 85 - DNA, 19 bp, Artificial Sequence
  Description of Artificial Sequence Synthetic oligonucleotide
aaatatttgctgtgtctca

SEQ ID NO: 86 - DNA, 19 bp, Artificial Sequence
  Description of Artificial Sequence Synthetic oligonucleotide
ctgtaagttcagggagaga

SEQ ID NO: 87 - DNA, 19 bp, Artificial Sequence
  Description of Artificial Sequence Synthetic oligonucleotide
ggagagattccaagaaaac

SEQ ID NO: 88 - DNA, 19 bp, Artificial Sequence
  Description of Artificial Sequence Synthetic oligonucleotide
ccaagaaaacagctataat

SEQ ID NO: 89 - DNA, 19 bp, Artificial Sequence
  Description of Artificial Sequence Synthetic oligonucleotide
gtcccagcccagtgaagta

SEQ ID NO: 90 - DNA, 19 bp, Artificial Sequence
  Description of Artificial Sequence Synthetic oligonucleotide
agaagtagcgcgacgtttt

SEQ ID NO: 91 - DNA, 19 bp, Artificial Sequence
  Description of Artificial Sequence Synthetic oligonucleotide
agtcaatggctcccacgaa

SEQ ID NO: 92 - DNA, 19 bp, Artificial Sequence
  Description of Artificial Sequence Synthetic oligonucleotide
gagggaaagccaagagagg

SEQ ID NO: 93 - DNA, 19 bp, Artificial Sequence
  Description of Artificial Sequence Synthetic oligonucleotide
tgaagtacagactgaagtt

SEQ ID NO: 94 - DNA, 19 bp, Artificial Sequence
  Description of Artificial Sequence Synthetic oligonucleotide
aggaaaactccatgcaagt

SEQ ID NO: 95 - DNA, 19 bp, Artificial Sequence
  Description of Artificial Sequence Synthetic oligonucleotide
gttcagggagagattccaa

SEQ ID NO: 96 - DNA, 19 bp, Artificial Sequence
  Description of Artificial Sequence Synthetic oligonucleotide
aacaagagagggaaagcca

SEQ ID NO: 97 - DNA, 19 bp, Artificial Sequence
  Description of Artificial Sequence Synthetic oligonucleotide
caagagagggaaagccaag

SEQ ID NO: 98 - DNA, 19 bp, Artificial Sequence
  Description of Artificial Sequence Synthetic oligonucleotide
gcagaagtagcgcgacgtt

SEQ ID NO: 99 - DNA, 19 bp, Artificial Sequence
  Description of Artificial Sequence Synthetic oligonucleotide
aaggaagcggctcggaaca

SEQ ID NO: 100 - DNA, 19 bp, Artificial Sequence
  Description of Artificial Sequence Synthetic oligonucleotide
gctcggaacatagcagttt

SEQ ID NO: 101 - DNA, 19 bp, Artificial Sequence
  Description of Artificial Sequence Synthetic oligonucleotide
gtaggaatacgaggagttt

SEQ ID NO: 102 - DNA, 19 bp, Artificial Sequence
  Description of Artificial Sequence Synthetic oligonucleotide
caaatttacggatgactgt

SEQ ID NO: 103 - DNA, 19 bp, Artificial Sequence
  Description of Artificial Sequence Synthetic oligonucleotide
gcgatccacagaactgaaa

SEQ ID NO: 104 - DNA, 19 bp, Artificial Sequence
  Description of Artificial Sequence Synthetic oligonucleotide
gagagggaaagccaagaga

SEQ ID NO: 105 - DNA, 20 bp, Artificial Sequence
  Description of Artificial Sequence Synthetic oligonucleotide
cataccactcccgccctgtt

SEQ ID NO: 106 - DNA, 21 bp, Artificial Sequence
  Description of Artificial Sequence Synthetic oligonucleotide
cataccactcccgccctgttt

SEQ ID NO: 107 - DNA, 19 bp, Artificial Sequence
  Description of Artificial Sequence Synthetic oligonucleotide
taccactcccgccctgttt

SEQ ID NO: 108 - DNA, 20 bp, Artificial Sequence
  Description of Artificial Sequence Synthetic oligonucleotide
ataccactcccgccctgttt

SEQ ID NO: 109 - DNA, 19 bp, Artificial Sequence
  Description of Artificial Sequence Synthetic oligonucleotide
ataccactcccgccctgtt

SEQ ID NO: 110 - DNA, 20 bp, Artificial Sequence
  Description of Artificial Sequence Synthetic oligonucleotide
gctttccctctcttgttcag

SEQ ID NO: 111 - DNA, 19 bp, Artificial Sequence
  Description of Artificial Sequence Synthetic oligonucleotide
gctttccctctcttgttca

SEQ ID NO: 112 - DNA, 21 bp, Artificial Sequence
  Description of Artificial Sequence Synthetic oligonucleotide
gctttccctctcttgttcagg

SEQ ID NO: 113 - DNA, 21 bp, Artificial Sequence
  Description of Artificial Sequence Synthetic oligonucleotide
ggctttccctctcttgttcag

SEQ ID NO: 114 - DNA, 20 bp, Artificial Sequence
  Description of Artificial Sequence Synthetic oligonucleotide
ggctttccctctcttgttca

SEQ ID NO: 115 - DNA, 21 bp, Homo sapiens
  CDS (1)..(21)
agcagtagcgctatgtcttcc

SEQ ID NO: 116 - protein, 7 aa, Homo sapiens
SSSAMSS

SEQ ID NO: 117 - DNA, 21 bp, Homo sapiens
  CDS (1)..(21)
agcagtagcgctgtcttcctc

SEQ ID NO: 118 - protein, 7 aa, Homo sapiens
SSSAVFL

(19) United States
(12) Patent Application Publication
(10) Pub. No.: US 2016/0009797 A1
(43) Pub. Date: Jan. 14, 2016

(54) METHODS FOR REGULATING HAIR GROWTH DISORDERS

(71) Applicant: THE TRUSTEES OF COLUMBIA UNIVERSITY IN THE CITY OF NEW YORK, New York, NY (US)

(72) Inventor: Angela M. Christiano, Mahwah, NJ (US)

(73) Assignee: THE TRUSTEES OF COLUMBIA UNIVERSITY IN THE CITY OF NEW YORK, New York, NY (US)

(21) Appl. No.: 14/609,691

(22) Filed: Jan. 30, 2015

Related U.S. Application Data
(63) Continuation of application No. PCT/US2013/053210, filed on Aug. 1, 2013.
(60) Provisional application No. 61/678,339, filed on Aug. 1, 2012.

(51) Int. Cl. C07K 16/22; G01N 33/74; C12Q 1/68; C12N 15/113

(52) U.S. Cl. CPC C07K 16/22; C12N 15/1136; G01N 33/74; C12Q 1/6883; C07K 2317/76; C12N 2310/14; C12N 2310/11; C12N 2320/30; C12Q 2600/156; C12Q 2600/158; G01N 2333/50; G01N 2800/50

(57) ABSTRACT

The presently disclosed subject matter provides methods for treating a hair loss disorder in a subject by administering a FGF5 inhibitor. The presently disclosed subject matter further provides methods for treating a hair growth disorder in a subject by administering a FGF5 activator.

### PRIORITY CLAIMED

This application is a continuatin of PCT Application No. PCT/US2013/053210, filed Aug. 1, 2013, and claims priority under 35 U.S.C. §119 to U.S. Application Ser. No. 61/678,339, filed Aug. 1, 2012, and the disclosures of which are hereby incorporated by reference in their entireties herein.

### SEQUENCE LISTING

The instant application contains a Sequence Listing which has been submitted in ASCII format via EFS-Web and is hereby incorporated by reference in its entirety. Said ASCII copy, created on Jan. 30, 2015, is named 0700505485_SeqList.txt, and is 44,669 bytes in size. The sequence listing submitted herewith, does not extend beyond the scope of the specification, and thus, does not contain new matter.

### BACKGROUND

Hair follicles pass through a three-stage cycle that comprises anagen (hair growth), catagen (the cessation of growth), and telogen (a resting phase); after telogen, the hair falls out of the follicle before a new cycle begins. Abnormal hair loss (alopecia, hypotrichosis) or abnormal hair growth (hypertrichosis, hirsutism) can be medically treated by targeting the mechanisms that regulate the hair growth cycle.

Alopecia Areata (AA) is one of the most highly prevalent autoimmune diseases, leading to hair loss due to the collapse of immune privilege of the hair follicle and subsequent autoimmune destruction. AA is a skin disease that leads to hair loss on the scalp and elsewhere. In some severe cases, it can progress to complete loss of hair on the head or body. Although AA is believed to be caused by autoimmunity, the gene level diagnosis and treatment are seldom reported. The genetic basis of AA is largely unknown.

Hypertrichosis is defined as excessive hair growth for a particular site of the body or age of a patient that is not hormone-dependent. Hypertrichoses are characterized on the basis of multiple criteria: cause (genetic or acquired), age of onset, extent of hair distribution (universal or localized) and affected sites. Several different forms of hypertrichosis in humans include, but are not limited to, X-linked hypertrichosis (OMIM 307150), generalized hypertrichosis terminalis with or without gingival hyperplasia (CGHT; OMIM 135400), autosomal recessive hypertrichosis, Cantu syndrome (OMIM 239850), Ambras type hypertrichosis (AS; OMIM 145701), and trichomegaly (e.g., ciliary or autosomal recessive trichomegaly (OMIM 190330)).

### SUMMARY

The presently disclosed subject matter provides for a method of treating a hair-loss disorder in a mammalian subject in need thereof. The method includes administering to the subject an inhibitor of FGF5. In one embodiment, the inhibitor includes an antibody that specifically binds to a protein comprising SEQ ID NO: 1 or 3. In some embodiments, the inhibitor is an antisense RNA that specifically inhibits expression of the gene that encodes the FGF5 protein; a siRNA that specifically targets the gene that encodes the FGF5 protein; or a small molecule. In other embodiments, the siRNA is directed to a human nucleic acid sequence comprising SEQ ID NO: 2 or 4. In further embodiments, he siRNA directed to a FGF5 gene is any one of the sequences listed in Table 1. In some embodiments, the antisense RNA specifically binds to a human nucleic acid sequence comprising SEQ ID NO: 2 or 4. In other embodiments, the antisense RNA includes SEQ ID NO: 105, 106, 107, 108, or 109. In another embodiment, the hair-loss disorder includes thinning eyelashes, androgenetic alopecia, telogen effluvium, alopecia areata, telogen effluvium, tinea capitis, alopecia totalis, hypotrichosis, hereditary hypotrichosis simplex, or alopecia universalis. In a further embodiment, the method further includes determining whether the inhibitor administered induced hair growth in the subject afflicted with a hair loss disorder as compared to the subject's hair growth prior to treatment with the inhibitor.

The presently disclosed subject matter further provides for a method for inducing hair growth in a subject in need thereof. The method includes administering to the subject an effective amount of an inhibitor of FGF5, thereby controlling hair growth in the subject. In one embodiment, the subject is afflicted with a hair-loss disorder. In one embodiment, the inhibitor includes an antibody that specifically binds to a protein comprising SEQ ID NO: 1 or 3. In some embodiments, the inhibitor is an antisense RNA that specifically inhibits expression of the gene that encodes the FGF5 protein; a siRNA that specifically targets the gene that encodes the FGF5 protein; or a small molecule. In other embodiments, the siRNA is directed to a human nucleic acid sequence comprising SEQ ID NO: 2 or 4. In further embodiments, he siRNA directed to a FGF5 gene is any one of the sequences listed in Table 1. In some embodiments, the antisense RNA specifically binds to a human nucleic acid sequence comprising SEQ ID NO: 2 or 4. In other embodiments, the antisense RNA includes SEQ ID NO: 105, 106, 107, 108, or 109. In another embodiment, the hair-loss disorder includes thinning eyelashes, androgenetic alopecia, telogen effluvium, alopecia areata, telogen effluvium, tinea capitis, alopecia totalis, hypotrichosis, hereditary hypotrichosis simplex, or alopecia universalis. In a further embodiment, the method further includes determining whether the inhibitor administered induced hair growth in the subject afflicted with a hair loss disorder as compared to the subject's hair growth prior to treatment with the inhibitor.

The presently disclosed subject matter also provides a method of treating a hair-growth disorder in a mammalian subject in need thereof, the method comprising administering to the subject an activator of FGF5. In one embodiment, the subject is afflicted with a hair-growth disorder. In another embodiment, the hair-growth disorder includes X-linked hypertrichosis, generalized hypertrichosis terminalis with gingival hyperplasia, generalized hypertrichosis terminalis, autosomal recessive hypertrichosis, Cantu syndrome, Ambras type hypertrichosis, or trichomegaly. In a further embodiment, the trichomegaly is ciliary trichomegaly or autosomal recessive trichomegaly. In another embodiment, the activator is a polypeptide comprising SEQ ID NO: 1 or 3, or a fragment thereof; or a peptidomimetic comprising SEQ ID NO: 1 or 3.

Additionally, the presently disclosed subject matter provides a method for reducing hair growth in a subject. The method includes administering to the subject an effective amount of an activator of FGF5, thereby controlling hair growth in the subject. In one embodiment, the subject is afflicted with a hair-growth disorder. In one embodiment, the subject is afflicted with a hair-growth disorder. In another embodiment, the hair-growth disorder includes X-linked hypertrichosis, generalized hypertrichosis terminalis with gingival hyperplasia, generalized hypertrichosis terminalis, autosomal recessive hypertrichosis, Cantu syndrome, Ambras type hypertrichosis, or trichomegaly. In a further embodiment, the trichomegaly is ciliary trichomegaly or autosomal recessive trichomegaly. In one embodiment, the method further includes determining whether the activator administered reduced hair growth in the subject afflicted with a hair-growth disorder as compared to the subject's hair growth prior to treatment with the activator. In another embodiment, the activator is a polypeptide comprising SEQ ID NO: 1 or 3, or a fragment thereof; or a peptidomimetic comprising SEQ ID NO: 1 or 3.

The presently disclosed subject matter provides a method for reducing hair growth in a subject. The method includes administering to the subject an effective amount of a FGF5 protein, thereby controlling hair growth in the subject. In one embodiment, the subject is afflicted with a hair-growth disorder. In another embodiment, the hair-growth disorder includes X-linked hypertrichosis, generalized hypertrichosis terminalis with gingival hyperplasia, generalized hypertrichosis terminalis, autosomal recessive hypertrichosis, Cantu syndrome, Ambras type hypertrichosis, or trichomegaly. In a further embodiment, the trichomegaly is ciliary trichomegaly or autosomal recessive trichomegaly. In one embodiment, the method further includes determining whether the FGF5 protein administered reduced hair growth in the subject afflicted with a hair-growth disorder as compared to the subject's hair growth prior to treatment with the protein.

The presently disclosed subject matter provides a method of treating a hair disorder in a mammalian subject in need thereof, the method includes administering to the subject a compound that modulates the expression of FGF5. In one embodiment, the hair disorder is a hair-loss disorder. In another embodiment, the hair-loss disorder includes thinning eyelashes, androgenetic alopecia, telogen effluvium, alopecia areata, telogen effluvium, tinea capitis, alopecia totalis, hypotrichosis, hereditary hypotrichosis simplex, or alopecia universalis. In one embodiment, the hair disorder is a hair-growth disorder. In another embodiment, the hair-growth disorder includes X-linked hypertrichosis, generalized hypertrichosis terminalis, generalized hypertrichosis terminalis with gingival hyperplasia, autosomal recessive hypertrichosis, Cantu syndrome, Ambras type hypertrichosis, or trichomegaly. In a further embodiment, the trichomegaly is ciliary trichomegaly or autosomal recessive trichomegaly. In some embodiments, the method further includes determining whether the compound administered induced hair growth in the subject afflicted with a hair-loss disorder as compared to the subject's hair growth prior to treatment with the compound. In other embodiments, the method further includes determining whether the compound administered reduced hair growth in the subject afflicted with a hair-growth disorder as compared to the subject's hair growth prior to treatment with the compound. In some embodiments, the compound is a polypeptide comprising SEQ ID NO: 1 or 3, or a fragment thereof; or a peptidomimetic comprising SEQ ID NO: 1 or 3. In another embodiment, the compound is an antisense RNA that specifically inhibits expression of the gene that encodes the FGF5 protein; a siRNA that specifically targets the gene that encodes the FGF5 protein; or a small molecule. In a further embodiment, the siRNA is directed to a human nucleic acid sequence comprising SEQ ID NO: 2 or 4. In some embodiments, the siRNA directed to a FGF5 gene is any one of the sequences listed in Table 1. In other embodiments, the antisense RNA specifically binds to a human nucleic acid sequence comprising SEQ ID NO: 2 or 4. In further embodiments, the antisense RNA includes SEQ ID NO: 105, 106, 107, 108, or 109. In yet other embodiments, the compound includes an antibody that specifically binds to a protein comprising SEQ ID NO: 1 or 3.

The presently disclosed subject matter provides a method for detecting the presence of or a predisposition to a hair-loss disorder in a human subject. The method includes (a) obtaining a biological sample from a human subject; and (b) detecting whether or not there is an alteration in the expression of FGF5 in the subject as compared to a subject not afflicted with a hair-loss disorder. In one embodiment, the hair-loss disorder includes thinning eyelashes, androgenetic alopecia, telogen effluvium, alopecia areata, telogen effluvium, tinea capitis, alopecia totalis, hypotrichosis, hereditary hypotrichosis simplex, or alopecia universalis. In another embodiment, the detecting includes detecting whether there is an alteration in the FGF5 gene locus. In some embodiments, the alteration includes a 2 bp deletion in exon 1 of the FGF5 gene. In other embodiments, the deletion includes a thymine and adenine at positions 159 and 160. In further embodiments, the detecting includes detecting whether expression of FGF5 is reduced. In some embodiments, the detecting includes detecting in the sample whether there is a reduction in FGF5 mRNA, FGF5 protein, or a combination thereof. In other embodiments, detecting includes gene sequencing, selective hybridization, amplification, gene expression analysis, or a combination thereof. In further embodiments, a reduction in FGF5 expression of at least 20% indicates a predisposition to or presence of a hair-loss disorder in the subject.

The presently disclosed subject matter provides a diagnostic kit for determining whether a sample from a subject exhibits reduced FGF5 expression or exhibits an FGF5 gene mutation. The kit includes nucleic acid primers that specifically hybridize to and prime a polymerase reaction from FGF5. In one embodiment, the mutation includes a 2 bp deletion in exon 1 of the FGF5 gene. In another embodiment, the deletion includes a thymine and adenine at positions 159 and 160, respectively. In a further embodiment, the primers are directed to SEQ ID NOS: 2 or 4.

### BRIEF DESCRIPTION OF THE FIGURES

FIG. 1A-C Phenotype and mutation in trichomegaly with hypertrichosis. FIG. 1A Patient shows trichomegaly with hypertrichosis on the face, eyebrows and eyelashes, as well as the arms. FIG. 1B A 2 bp deletion in patients results in a frame shift within FGF5. FIG. 1C FGF5 and FGF5S are present within the outer root sheath surrounding plucked hair fibers, but are absent in fibers obtained from patients.

FIG. 2 shows the sequence of the 2 isoforms of FGF5.

FIG. 3A-D are photographic images of clinical manifestations of AA. In the upper panels (FIGS. 3A-B), patients with AA multiplex. In FIG. 3B, the patient is in regrowth phase. For patients with alopecia universalis, there is a complete lack of body hair and scalp hair (FIG. 3C), while patients with alopecia totalis only lack scalp hair (FIG. 3D). In FIG. 3D, hair regrowth is observed in the parietal region, while no regrowth in either occipital or temporal regions is evident.

### DETAILED DESCRIPTION

While over the counter remedies are available to induce hair growth, they are only effective in a subset of patients and result in temporary hair growth. A therapeutic that is more efficacious and directed is needed. This technology identifies mutations in FGF5 in humans that have trichomegaly (excessively long eyelashes) and hypertrichosis (too much hair). The work described in Example 1 is the first to link the resulting loss-of-expression of FGF5 to the hair overgrowth phenotype in a human. This finding can be used to develop new directed inhibitors against FGF5 to induce eyelash growth for patients with alopecia, thin lashes, or hypotrichosis. This could also be used to develop agonists of FGF5 to reduce hair growth in patients with hypertrichosis.

Fibroblast Growth Factor 5 (FGF5) is a protein that normally suppresses/inhibits hair growth in mammals by inducing the transition from anagen to catagen. FGF is expressed in the hair follicle (e.g., see Example 1 for first reports in human and mouse studies by Hebert et al., ((1994) Cell, September 23; 78(6):1017-25)). Inhibition of this protein's action or suppression of its expression via a mutation is associated with excessive hair growth in mice and other animals (e.g., Hebert et al, (1994) Cell, 78(6):1017-25; Mizuno et al, (2011) Exp Anim. 60(2):161-7.). Under-expression of this protein in various mammals has been linked to abnormally excessive hair growth and elongation of anagen. FGF5 is also associated with coat-length variation in dogs (Cadieu et al, Science. 2009 October 2; 326(5949):150-3) and in cats (Kehler, et al, J Hexed. 2007 September-October; 98(6):555-66.), as well as in goats and rabbits.

The presently disclosed subject matter provides that the under-expression of FGF5 can be causally linked to the excessive hair growth, thus indicating that pharmacological inhibition of FGF5 can increase hair growth. Mutations causing loss of expression of FGF5 have been linked to excessive hair growth in humans with trichomegaly and hypertrichosis, which can be exploited to modulate abnormal hair growth by altering the expression or activity of FGF5 in terminal hair regions such as the scalp. As discussed in Example 1, the inventor has identified a mutation in the FGF5 gene in a family with trichomegaly (excessively long eyelashes) and hypertrichosis (too much hair). The inventors show a that under-expression of FGF5 is causally linked to the excessive hair growth, raising the possibility that pharmacological inhibition of FGF5 may increase hair growth.

The presently disclosed subject matter thus provides for methods of treating a hair loss disorder (e.g., thinning eye lashes or scalp hair loss) with an inhibitor of FGF5. The presently disclosed subject matter provides for therapeutics previously untested in hair loss, that can inform one about the clinical relevance of this pathway in hair loss and related diseases. The presently disclosed subject matter further provides for methods of treating a hair growth disorder, such as hypertrichosis, with an activator of FGF5. In one embodiment, inhibition of FGF5 activity or expression via targeted gene therapy can increase hair growth in patients afflicted with alopecia or hypotrichosis. In another embodiment, excitation of FGF5 activity or expression can reduce hair growth in patients afflicted with hypertrichosis or hirsutism.

In some embodiments, the inhibition or activation of FGF5 (e.g., expression or activity) can be employed as part of an in vivo gene therapy for restoration of normal hair growth in patients suffering from insufficient or excessive hair growth (for hair disorders, see J. Invest. Derm. Symp. Proc. 2003, Vol. 8, pp. 204-206). Since manipulation of FGF5 expression or activity does not alter the metabolism of androgenic hormones, hair growth therapies based upon FGF5 modulation can obviate some of the side effects and/or risks associated with androgen inhibitors. Modulating FGF5 expression or activity in conjunction with therapies that target other factors that control the hair growth cycle can synergistically improve the effects of existing treatments for hair growth problems.

Overview of the Integument and Hair Cells

The integument (or skin) is the largest organ of the body and is a highly complex organ covering the external surface of the body. It merges, at various body openings, with the mucous membranes of the alimentary and other canals. The integument performs a number of essential functions such as maintaining a constant internal environment via regulating body temperature and water loss; excretion by the sweat glands; but predominantly acts as a protective barrier against the action of physical, chemical and biologic agents on deeper tissues. Skin is elastic and except for a few areas such as the soles, palms, and ears, it is loosely attached to the underlying tissue. It also varies in thickness from 0.5 mm (0.02 inches) on the eyelids (“thin skin”) to 4 mm (0.17 inches) or more on the palms and soles (“thick skin”) (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The skin is composed of two layers: a) the epidermis and b) the dermis. The epidermis is the outer layer, which is comparatively thin (0.1 mm). It is several cells thick and is composed of 5 layers: the stratum germinativum, stratum spinosum, stratum granulosum, stratum lucidum (which is limited to thick skin), and the stratum corneum. The outermost epidermal layer (the stratum corneum) consists of dead cells that are constantly shed from the surface and replaced from below by a single, basal layer of cells, called the stratum germinativum. The epidermis is composed predominantly of keratinocytes, which make up over 95% of the cell population. Keratinocytes of the basal layer (stratum germinativum) are constantly dividing, and daughter cells subsequently move upwards and outwards, where they undergo a period of differentiation, and are eventually sloughed off from the surface. The remaining cell population of the epidermis includes dendritic cells such as Langerhans cells and melanocytes. The epidermis is essentially cellular and non-vascular, containing little extracellular matrix except for the layer of collagen and other proteins beneath the basal layer of keratinocytes (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The dermis is the inner layer of the skin and is composed of a network of collagenous extracellular material, blood vessels, nerves, and elastic fibers. Within the dermis are hair follicles with their associated sebaceous glands (collectively known as the pilosebaceous unit) and sweat glands. The interface between the epidermis and the dermis is extremely irregular and uneven, except in thin skin. Beneath the basal epidermal cells along the epidermal-dermal interface, the specialized extracellular matrix is organized into a distinct structure called the basement membrane (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The mammalian hair fiber is composed of keratinized cells and develops from the hair follicle. The hair follicle is a peg of tissue derived from a downgrowth of the epidermis, which lies immediately underneath the skin's surface. The distal part of the hair follicle is in direct continuation with the external, cutaneous epidermis. Although a small structure, the hair follicle comprises a highly organized system of recognizably different layers arranged in concentric series. Active hair follicles extend down through the dermis, the hypodermis (which is a loose layer of connective tissue), and into the fat or adipose layer (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

At the base of an active hair follicle lies the hair bulb. The bulb consists of a body of dermal cells, known as the dermal papilla, contained in an inverted cup of epidermal cells known as the epidermal matrix. Irrespective of follicle type, the germinative epidermal cells at the very base of this epidermal matrix produce the hair fiber, together with several supportive epidermal layers. The lowermost dermal sheath is contiguous with the papilla basal stalk, from where the sheath curves externally around all of the hair matrix epidermal layers as a thin covering of tissue. The lowermost portion of the dermal sheath then continues as a sleeve or tube for the length of the follicle (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

Developing skin appendages, such as hair and feather follicles, rely on the interaction between the epidermis and the dermis, the two layers of the skin. In embryonic development, a sequential exchange of information between these two layers supports a complex series of morphogenetic processes, which results in the formation of adult follicle structures. However, in contrast to general skin dermal and epidermal cells, certain hair follicle cell populations, following maturity, retain their embryonic-type interactive, inductive, and biosynthetic behaviors. These properties can be derived from the very dynamic nature of the cyclical productive follicle, wherein repeated tissue remodeling necessitates a high level of dermal-epidermal interactive communication, which is vital for embryonic development and would be desirable in other forms of tissue reconstruction.

The hair fiber is produced at the base of an active follicle at a very rapid rate. For example, follicles produce hair fibers at a rate 0.4 mm per day in the human scalp and up to 1.5 mm per day in the rat vibrissa or whiskers, which means that cell proliferation in the follicle epidermis ranks amongst the fastest in adult tissues (Malkinson F D and J T Kearn, Int J Dermatol 1978, 17:536-551). Hair grows in cycles. The anagen phase is the growth phase, wherein up to 90% of the hair follicles said to be in anagen; catagen is the involuting or regressing phase which accounts for about 1-2% of the hair follicles; and telogen is the resting or quiescent phase of the cycle, which accounts for about 10-14% of the hair follicles. The cycle's length varies on different parts of the body.

Hair follicle formation and cycling is controlled by a balance of inhibitory and stimulatory signals. The signaling cues are potentiated by growth factors that are members of the TGF-BMP family. A prominent antagonist of the members of the TGF-BMP family is follistatin. Follistatin is a secreted protein that inhibits the action of various BMPs (such as BMP-2, -4, -7, and -11) and activins by binding to said proteins, and purportedly plays a role in the development of the hair follicle (Nakamura M, et al., FASEB J, 2003, 17(3):497-9; Patel K Intl J Biochem Cell Bio, 1998, 30:1087-93; Ueno N, et al., PNAS, 1987, 84:8282-86; Nakamura T, et al., Nature, 1990, 247:836-8; Iemura S, et al., PNAS, 1998, 77:649-52; Fainsod A, et al., Mech Dev, 1997, 63:39-50; Gamer L W, et al., Dev Biol, 1999, 208:222-32).

The deeply embedded end bulb, where local dermal-epidermal interactions drive active fiber growth, is the signaling center of the hair follicle comprising a cluster of mesenchymal cells, called the dermal papilla (DP). This same region is also central to the tissue remodeling and developmental changes involved in the hair fiber's or appendage's precise alternation between growth and regression phases. The DP, a key player in these activities, appears to orchestrate the complex program of differentiation that characterizes hair fiber formation from the primitive germinative epidermal cell source (Oliver R F, J Soc Cosmet Chem, 1971, 22:741-755; Oliver R F and C A Jahoda, Biology of Wool and Hair (eds Roger et al.), 1971, Cambridge University Press: 51-67; Reynolds A J and C A Jahoda, Development, 1992, 115:587-593; Reynolds A J, et al., J Invest Dermatol, 1993, 101:634-38).

The lowermost dermal sheath (DS) arises below the basal stalk of the papilla, from where it curves outwards and upwards. This dermal sheath then externally encases the layers of the epidermal hair matrix as a thin layer of tissue and continues upward for the length of the follicle. The epidermally-derived outer root sheath (ORS) also continues for the length of the follicle, which lies immediately internal to the dermal sheath in between the two layers, and forms a specialized basement membrane termed the glassy membrane. The outer root sheath constitutes little more than an epidermal monolayer in the lower follicle, but becomes increasingly thickened as it approaches the surface. The inner root sheath (IRS) forms a mold for the developing hair shaft. It comprises three parts: the Henley layer, the Huxley layer, and the cuticle, with the cuticle being the innermost portion that touches the hair shaft. The IRS cuticle layer is a single cell thick and is located adjacent to the hair fiber. It closely interdigitates with the hair fiber cuticle layer. The Huxley layer can comprise up to four cell layers. The IRS Henley layer is the single cell layer that runs adjacent to the ORS layer (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

As discussed herein, hair is formed by cell replication within hair follicles, where hair follicles produce either fetal hair, vellus hair, or terminal hair. Examples of terminal hair include eyelashes, eyebrows, scalp hair, and pubic hair (Elder (1997) Opth. Plas. Reconst. Surg., 13: 21-25). Eyelashes are the first terminal hairs to appear during embryological development. The structure of an eyelash is very close to that of a scalp hair fiber. The eyelash has 3 compartments, like scalp hair: the cuticle as the outermost compartment (with about 7 overlapping cell layers), the cortex, and the inner-most layer, the medulla. However, the eyelash is much shorter due to a shorter hair cycle. The epidermis surrounding an eyelash hair follicle is much thinner and has no hypodermis, compared to the epidermis of the scalp (Thibaut et al., (2010) Br J Derm., 162:304-310). The overall structure of the eyelash hair follicle is also similar to that of a scalp hair follicle, except that the eyelash hair follicle is much shorter and rooted about 2 mm deep into the dermis (Thibaut et al., (2010) Br J Derm., 162:304-310).

Hair growth mechanisms are dependent on anatomic location. The eylash gows about 14-150 μM daily and its growth cycle lasts about 5 to 6 months—about 30 days in anagen, about 15 days in catagen, and about over 100 days in telogen (Paul et al., (2012) Int. J Derm., 51:631-646; Thibaut et al., (2010) Br J Derm., 162:304-310). In comparison to scalp hair, eyelashes have a low ratio of hair follicles in anagen as compared to hair follicles in telogen—approximately 50% of eyelashes are in the anagen phase at all times as compared to 85-90% of scalp hairs (Paul et al., (2012) Int. J Derm., 51:631-646; Thibaut et al., (2010) Br J Derm., 162:304-310). The duration of anagen for an eyelash hair follicle is much shorter than those hair follicles found in the scalp.

Eyelash (or ciliary) trichomegaly refers to eyelashes which are found to be of increased length, thickness, and pigmentation. Eyelash trichomegaly has been defined as eyelashes measuring greater than 12 mm in length, which are deemed clinically and abnormally long (Paul et al., (2012) Int. J Derm., 51:631-646).

Hypertrichosis syndromes fall under the larger umbrella of ectodermal dysplasias, which are characterized by abnormal development of the hair, skin, nails, teeth and/or eccrine glands. While these appendages vary greatly in their shape and function, they share several common developmental features, namely, formation through a series of interactions between the epithelia and adjacent mesenchyme during embryogenesis. Interestingly, many additional anomalies are associated with hypertrichosis. Members of the X-linked hypertrichosis family identified also exhibit dental anomalies and deafness. Moreover, generalized hypertrichosis terminalis is often associated with gingival hyperplasia; Cantu syndrome is additionally characterized by skeletal dysplasia and cardiomegaly; and Ambras syndrome patients commonly present with facial dysmorphologies and bone abnormalities. Despite the wide range phenotypes of these syndromes, the causative mechanism(s) underlying many forms of human hypertrichoses remain unknown.

Treatment of Hair Loss Disorders

This presently disclosed subject matter provides for the discovery that an inhibitor of FGF5 can be used for the treatment of hair loss disorders. Non-limiting examples of hair loss disorders include: thinning eyelashes, androgenetic alopecia, Alopecia areata, telogen effluvium, alopecia areata, alopecia totalis, and alopecia universalis.

The presently disclosed subject matter encompasses a method of treating a hair-loss disorder in a mammalian subject in need thereof, the method comprising administering to the subject an inhibitor of FGF5. In one embodiment, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis. In one embodiment, the method further comprises determining whether the inhibitor administered induced hair growth in the subject afflicted with a hair loss disorder as compared to the subject's hair growth prior to treatment with the inhibitor. In one embodiment, the inhibitor comprises an antibody that specifically binds to a protein comprising SEQ ID NO: 1 or 3. In another embodiment, the inhibitor is an antisense RNA that specifically inhibits expression of the gene that encodes the FGF5 protein; a siRNA that specifically targets the gene that encodes the FGF5 protein, or a small molecule. In one embodiment, the siRNA is directed to a human nucleic acid sequence comprising SEQ ID NO: 2 or 4. In another embodiment, the siRNA directed to a FGF5 gene is any one of the sequences listed in Table 1. In a further embodiment, the antisense RNA that specifically inhibits expression of the gene that encodes the FGF5 protein is any one of the sequences listed in Table 3.

The presently disclosed subject matter also encompasses a method for inducing hair growth in a subject, the method comprising administering to the subject an effective amount of an inhibitor of FGF5, thereby controlling hair growth in the subject. In one embodiment, the subject is afflicted with a hair-loss disorder. In another embodiment, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis. In one embodiment, the method further comprises determining whether the inhibitor administered induced hair growth in the subject afflicted with a hair loss disorder as compared to the subject's hair growth prior to treatment with the inhibitor. In one embodiment, the inhibitor comprises an antibody that specifically binds to a protein comprising SEQ ID NO: 1 or 3. In another embodiment, the inhibitor is an antisense RNA that specifically inhibits expression of the gene that encodes the FGF5 protein; a siRNA that specifically targets the gene that encodes the FGF5 protein, or a small molecule. In one embodiment, the siRNA is directed to a human nucleic acid sequence comprising SEQ ID NO: 2 or 4. In another embodiment, the siRNA directed to a FGF5 gene is any one of the sequences listed in Table 1. In a further embodiment, the antisense RNA that specifically inhibits expression of the gene that encodes the FGF5 protein is any one of the sequences listed in Table 3.

Treatment of Hair Growth Disorders

The presently disclosed subject matter encompasses a method of treating a hair-growth disorder in a mammalian subject in need thereof, the method comprising administering to the subject an activator of FGF5. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and trichomegaly (e.g., ciliary or autosomal recessive trichomegaly). In one embodiment, the activator is a polypeptide comprising SEQ ID NO: 1 or 3, or a fragment thereof; or a peptidomimetic comprising SEQ ID NO: 1 or 3.

The presently disclosed subject matter further encompasses a method for reducing hair growth in a subject, the method comprising administering to the subject an effective amount of an activator of FGF5, thereby controlling hair growth in the subject. In one embodiment, the subject is afflicted with a hair-growth disorder. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and trichomegaly (e.g., ciliary or autosomal recessive trichomegaly). In another embodiment, the method further comprises determining whether the activator administered reduced hair growth in the subject afflicted with a hair-growth disorder as compared to the subject's hair growth prior to treatment with the activator. In one embodiment, the activator is a polypeptide comprising SEQ ID NO: 1 or 3, or a fragment thereof; or a peptidomimetic comprising SEQ ID NO: 1 or 3.

The presently disclosed subject matter encompasses a method for reducing hair growth in a subject, the method comprising administering to the subject an effective amount of a FGF5 protein, thereby controlling hair growth in the subject. In one embodiment, the subject is afflicted with a hair-growth disorder. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and trichomegaly (e.g., ciliary or autosomal recessive trichomegaly). In another embodiment, the method further comprises determining whether the FGF5 protein administered reduced hair growth in the subject afflicted with a hair-growth disorder as compared to the subject's hair growth prior to treatment with the protein. In one embodiment, the activator is a polypeptide comprising SEQ ID NO: 1 or 3, or a fragment thereof; or a peptidomimetic comprising SEQ ID NO: 1 or 3.

Treatment of Hair Disorders

The presently disclosed subject matter encompasses a method of treating a hair disorder in a mammalian subject in need thereof, the method comprising administering to the subject a compound that modulates the expression of FGF5.

The term “modulates,” as it appears herein, refers to a change in the activity or expression of a gene or protein of FGF5. For example, modulation can cause an increase or a decrease in protein activity, binding characteristics, or any other biological, functional, or immunological properties of a FGF5 protein.

In one embodiment, the hair disorder is a hair-loss disorder. In one embodiment, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis. In another embodiment, the hair disorder is a hair-growth disorder. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and trichomegaly (e.g., ciliary or autosomal recessive trichomegaly). In one embodiment, the method further comprises determining whether the compound administered induced hair growth in the subject afflicted with a hair loss disorder as compared to the subject's hair growth prior to treatment with the compound. In another embodiment, the method further comprises determining whether the compound administered reduced hair growth in the subject afflicted with a hair-growth disorder as compared to the subject's hair growth prior to treatment with the compound.

In a further embodiment, the administering comprises a subcutaneous, intra-muscular, intra-peritoneal, or intravenous injection; an infusion; oral, nasal, or topical delivery; or a combination thereof. In some embodiments, the administering occurs daily, weekly, twice weekly, monthly, twice monthly, or yearly.

DNA and Amino Acid Manipulation Methods and Purification Thereof

The practice of aspects of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual, 3^{rd} Ed., ed. by Sambrook (2001), Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture Of Animal Cells (R. I. Freshney, Alan R. Liss, Inc., 1987); Immobilized Cells and Enzymes (IRL Press, 1986); B. Perbal, A Practical Guide To Molecular Cloning (1984); the series, Methods In Enzymology (Academic Press, Inc., N.Y.), specifically, Methods In Enzymology, Vols. 154 and 155 (Wu et al. eds.); Gene Transfer Vectors For Mammalian Cells (J. H. Miller and M. P. Calos eds., 1987, Cold Spring Harbor Laboratory); Immunochemical Methods In Cell And Molecular Biology (Caner and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N. Y., 1986). All patents, patent applications and references cited herein are incorporated by reference in their entireties.

One skilled in the art can obtain a protein in several ways, which include, but are not limited to, isolating the protein via biochemical means or expressing a nucleotide sequence encoding the protein of interest by genetic engineering methods.

A protein is encoded by a nucleic acid (including, for example, genomic DNA, complementary DNA (cDNA), synthetic DNA, as well as any form of corresponding RNA). For example, it can be encoded by a recombinant nucleic acid of a gene. The proteins of the presently disclosed subject matter can be obtained from various sources and can be produced according to various techniques known in the art. For example, a nucleic acid that encodes a protein can be obtained by screening DNA libraries, or by amplification from a natural source. A protein can be a fragment or portion thereof. The nucleic acids encoding a protein can be produced via recombinant DNA technology and such recombinant nucleic acids can be prepared by conventional techniques, including chemical synthesis, genetic engineering, enzymatic techniques, or a combination thereof. For example, a FGF5 protein is the polypeptide encoded by the nucleic acid having the nucleotide sequence shown in SEQ ID NO: 2. An example of a FGF5 polypeptide has the amino acid sequence shown in SEQ ID NO: 1.

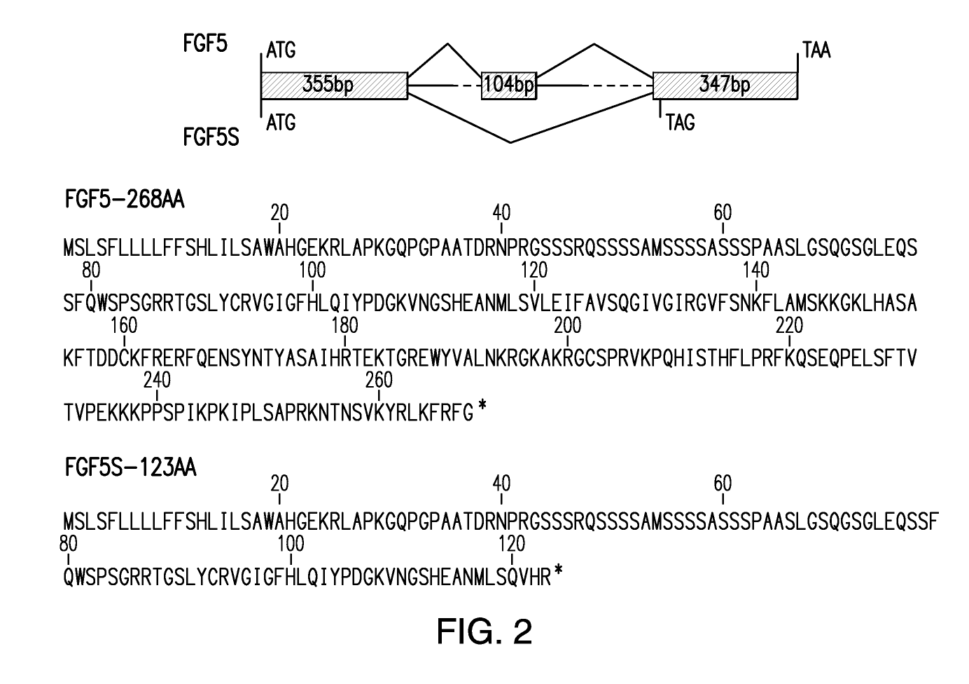

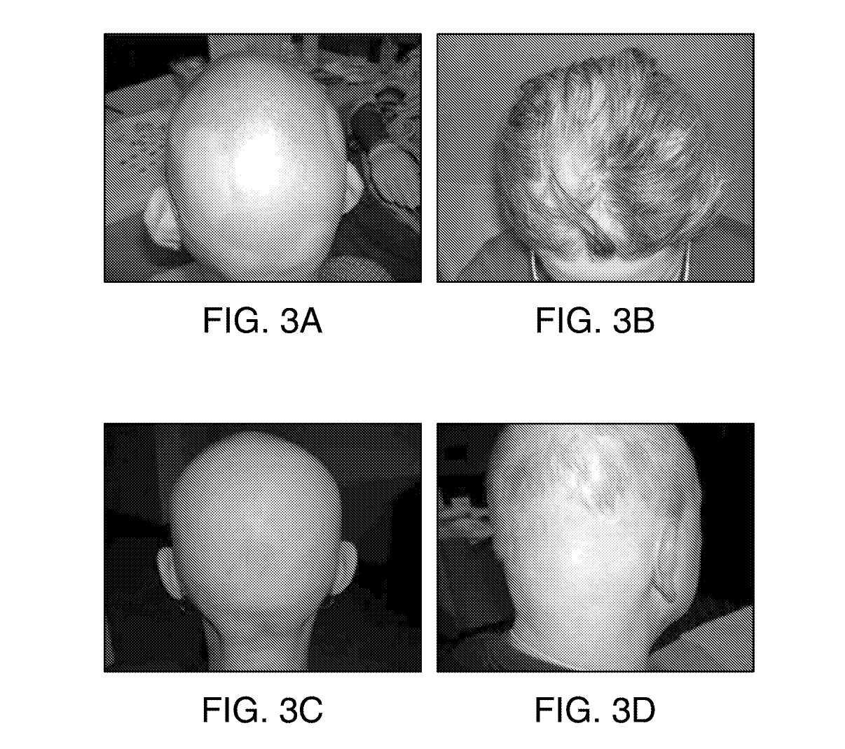

The polypeptide sequence of human FGF5 (transcript variant 1) is depicted in SEQ ID NO: 1. The nucleotide sequence of human FGF5 (transcript variant 1) is shown in SEQ ID NO: 2. Sequence information related to FGF5 (transcript variant 1) is accessible in public databases by GenBank Accession numbers NP_{—}004455 (protein) and NM_{—}004464 (nucleic acid). The polypeptide sequence is also depicted in FIG. 2.

SEQ ID NO: 1 is the human wild type amino acid sequence corresponding to FGF5 isoform 1 (residues 1-268):

| | |
|---|---|
| 1 | MSLSFLLLLF FSHLILSAWA HGEKRLAPKG QPGPAATDRN |
| | PRGSSSRQSS SSAMSSSSAS |
| 61 | SSPAASLGSQ GSGLEQSSFQ WSPSGRRTGS LYCRVGIGFH |
| | LQIYPDGKVN GSHEANMLSV |
| 121 | LEIFAVSQGI VGIRGVFSNK FLAMSKKGKL HASAKFTDDC |
| | KFRERFQENS YNTYASAIHR |
| 181 | TEKTGREWYV ALNKRGKAKR GCSPRVKPQH ISTHFLPRFK |
| | QSEQPELSFT VTVPEKKKPP |
| 241 | SPIKPKIPLS APRKNTNSVK YRLKFRFG |

SEQ ID NO: 2 is the human wild type nucleotide sequence corresponding to FGF5 (transcript variant 1) (nucleotides 1-5399), wherein the underscored bolded “ATG” denotes the beginning of the open reading frame:

| | |
|---|---|
| 1 | ggggaagctt cgcaggcgtg cacggagcag tgagatcact ggcgttataa atatcccggt |
| 61 | gccagcgcgg agatccgctc gggtggcctc tctcttcccc tctccccttc tcttccccga |
| 121 | ggctatgtcc acccggtgcg gcgaggcggg cagagccaga ggcacgcagc cgcacagggg |
| 181 | ctacagagcc cagaatcagc cctacaagat gcacttagga cccccgcggc tggaagaatg |
| 241 | agcttgtcct tcctcctcct cctcttcttc agccacctga tcctcagcgc ctgggctcac |
| 301 | ggggagaagc gtctcgcccc caaagggcaa cccggacccg ctgccactga taggaaccct |
| 361 | agaggctcca gcagcagaca gagcagcagt agcgctatgt cttcctcttc tgcctcctcc |
| 421 | tcccccgcag cttctctggg cagccaagga agtggcttgg agcagagcag tttccagtgg |
| 481 | agcccctcgg ggcgccggac cggcagcctc tactgcagag tgggcatcgg tttccatctg |
| 541 | cagatctacc cggatggcaa agtcaatgga tcccacgaag ccaatatgtt aagtgttttg |
| 601 | gaaatatttg ctgtgtctca ggggattgta ggaatacgag gagttttcag caacaaattt |
| 661 | ttagcgatgt caaaaaaagg aaaactccat gcaagtgcca agttcacaga tgactgcaag |
| 721 | ttcagggagc gttttcaaga aaatagctat aatacctatg cctcagcaat acatagaact |
| 781 | gaaaaaacag ggcgggagtg gtatgtggcc ctgaataaaa gaggaaaagc caaacgaggg |
| 841 | tgcagccccc gggttaaacc ccagcatatc tctacccatt ttctgccaag attcaagcag |
| 901 | tcggagcagc cagaactttc tttcacggtt actgttcctg aaaagaaaaa gccacctagc |
| 961 | cctatcaagc caaagattcc cctttctgca cctcggaaaa ataccaactc agtgaaatac |
| 1021 | agactcaagt ttcgctttgg ataatattcc tcttggcctt gtgagaaacc attctttccc |
| 1081 | ctcaggagtt tctataggtg tcttcagagt tctgaagaaa aattactgga cacagcttca |
| 1141 | gctatactta cactgtattg aagtcacgtc atttgtttca atgtgactga aacaaaatgt |
| 1201 | tttttgatag gaaggaaact ggaattcttt gtactaatac agggagcaca ctccttcagt |
| 1261 | tcagcaagac ataaagcctt ttgctttatg cttgagggat atttagaact ttgtattttc |
| 1321 | ggaaagttaa ataacaggga ctacgtattt ttctgacttt tacagattaa cctgaaagaa |
| 1381 | catacatgat acatttttat ttttggtttc caaagaatat tttgatgcag ataaaatatt |
| 1441 | ttgttaactt ttgttttttt ttgtttgttt tcttaaaagt acctctgcat tgagcatatt |
| 1501 | ttcttacttt tattatttta attaatatga cataagcaat cattttatgc tgtttatgaa |
| 1561 | ttataaatgt gtttatagct catttgtaat atggaaatct tttacatttt tcctattcac |
| 1621 | tgcacttttt tattgttttt atttctagcc atacctcaga taatatgttt agttttacat |
| 1681 | tttaaaatgt ttaaattctc tttcacagca ccaaaggctc agcttggatt tgtgtgtatg |
| 1741 | tgtatgtcaa ttcatgacat tatgtggaat cctaaacctt tggtggctgg gatatgatgg |
| 1801 | gttagaagca aggagaaaat ataaggactt tttgatggaa ttaaatgtgg gaggtaagga |
| 1861 | aaaggattta gaggtaaaag tacactaagt ttgcaacatt tattgagatc taagtctgtc |
| 1921 | ttgccttcat ttctcttttt atctccccct tgccctcatt cttgaacagc tggaggaata |
| 1981 | cattttattc tgtccatgaa gcatacacta tgaaattcaa gtgcttaaaa atacttctat |
| 2041 | gactctctgc tatcccactg tatagatcca cagggagcaa acacttagaa atgatagaga |
| 2101 | actgaaggag atcaatggtt taacagttat ccatgccaag tcccattgtc agaaatattc |
| 2161 | ttattactca gtcaaacact ctttgagctt cccttcctaa aggtaaccaa tccagtgaat |
| 2221 | agatgtgccc ttttataagg aaacttctga tgtttattaa aaaaactggc cttttgatag |
| 2281 | aggtaactta atttgggaat ttgttgtgtt gaaatggcat ttaatttcaa cctaaatact |
| 2341 | gactgctgga cataaatcac agaaaattta acttaagaaa atttacaaaa tttattctca |
| 2401 | ggtaatcatt ttaataaagt tctgcaaaat acacgtttat cttacattca gaaatgtggc |
| 2461 | aaaaaaggca tagctaaagg ctaaacatat ggctttagta gtaacaaaag ggttcataga |
| 2521 | aacttcatgg tttgcattta aacatgttta aagtgtactt ataaactatt tttttcttaa |
| 2581 | agcaaactat gatttatttt ggtgcacaaa tacaaagtgg aaacttacca aaattgaact |
| 2641 | agctaccata taagcagatt gctttaattt gatgggaaaa tagtacacac atatatataa |
| 2701 | caaataatat attaaaaaac ccatccatca actaaaacat tatatgtata catcagtata |
| 2761 | gtgttttatt ataaagccaa ttatctgatt aagcattctt tccactgaat gcataatgtt |
| 2821 | taaatagcat aaaatgaaat gctacaaaaa ttgaactaat ttatacttta aagtatttct |
| 2881 | gggttaaatg aaacaatgaa attttttagt atgttcaact ctcatccaaa tggcatatga |
| 2941 | ccctgtttac acagcctaaa gctaaaaata ttactctagt ttattctaat ctattgttaa |
| 3001 | gtattgtgca ctgtatacca agttcttagg gcacatgaaa aattttagct gccaaacagg |
| 3061 | aactagtaaa catatgttcc taataagtga agggaaagat aataatgatg gtcaacaata |
| 3121 | agccacgtca atgcataagt tgtataggct aaatgttgct tgtaggctac attaaactca |
| 3181 | aatgtaatag tttatcttat actcctggtt tgatttgatt agcatattaa cgtgaaagta |
| 3241 | ggatagctac taaatatata ttatgcaagt caggaatcat taatttcaaa atttaaagcc |
| 3301 | atgctaaaat taaaaagaaa atattaaatt acacaattac acttgtcttt actggccata |
| 3361 | caaaatgatt tttttttttt ttttgagaca gagtcttgct ctgtcaccag gctggagtgc |
| 3421 | agtggcatga tctcggctca ctgcaacctc caactccctg gtttaaggga ttctcctgcc |
| 3481 | tcagcctccc aagtagctgg gattacagac tcatgccacc acgccagcta atttttgtat |
| 3541 | ttttagtaga gacggggttt caccatgttg gtcaggatgg tctcaatcct ggcctcttga |
| 3601 | tagtcctgac ctcatgatct gcccacctcg gcctccccaa agtgctggga ttacaggtac |
| 3661 | aatgatgtat aattaatgct tagtgaagca taaagttacc tacatcaatt aattaaatga |
| 3721 | acttatgtac agaaaacatg tataaatata agtctatact aatgcttaca actttctaag |
| 3781 | agggttcttg cttatgtagc tttttattat tttaagtaac tagaaccacc aaatatcaaa |
| 3841 | taaaattatt tggttatggt tatgttcatc taaacacaac aataactttt atattaatat |
| 3901 | ttaggagtct attttgtcta taggtgacaa acatctccag actaacatgt cagttttatc |
| 3961 | aattatatta tgtttaatta tttaagattt ctttatgtgg aacatctata gagataaata |
| 4021 | gaaattttca ataagatgta gtaacactgt gatttatctt tcaagagtct ctcttcactt |
| 4081 | ccttctaaag agactaattt gagagtacag gtgcatatta attttcttgg ttctttcagc |
| 4141 | tgaattatat tggtccagaa gttcaaaatc atgtgacaat aataagggat actgacagaa |
| 4201 | gttatttcca agtttgtgta tatattataa aaattacata tataaaacta aggcttttat |
| 4261 | ttctgttatt tttaagcttt tatttcttgt agctaaaaat aaaacatcat aaatctggta |
| 4321 | ggtaaatttc ttattaaatc aatcttgaaa tagaaaatgt aataactttc ttaccattaa |
| 4381 | cattttttac ccttccatag aagggaggga ataaatcatg acttatccca ttttcaataa |
| 4441 | caaaacgaaa ctatggcact aaccaaaaac ttgcattctg gcataatttt tacagttgca |
| 4501 | gagaattgtt tctgggctca ttaaaaaaag tagtattgca gacattgctg caatgggaag |
| 4561 | cagacaataa cttcttaaag gaattctaca cctcctttaa gatttactta attgctacat |
| 4621 | ctaaattctg ataatttaaa atccatttta ggtgataaaa ttttttaaaa gttttgaagg |
| 4681 | aaacctctgg ataaatggac aaggcctaat ttttttttgt agtcaatcca actgtactgg |
| 4741 | ccaatttttg aaataagatt atatgattag gtattagcag agacaaagag ttacctcctc |
| 4801 | catcttactc tgccctattt gaaagtctca ggggagaaaa gggaacaaga tgctgatcca |
| 4861 | acctgagtgg agtcaggtga ggcatcttta catctaagaa ttttttttta aattttatta |
| 4921 | ttattatact tcaagttcta gggtacatgt ccacaatgca catgtctgtc acacatgcac |
| 4981 | acatgtgcca tgctggtgtg ctgcacccac caacctgtca tccagcatta ggtatatctc |
| 5041 | ctaatgctat ccctcccctc tccacccacc ccacagcagg ccccggtatg tgatgttccc |
| 5101 | cttcgtgtgt ccatgtgttc ttattgttca attcccacct atgagtgaga atatgtggtg |
| 5161 | tttggttttt ggtccttgca atagtttgct gagaatgatg gtttccagct tcatccatgt |
| 5221 | ccctacaaag aacatgaact catcattttt tatggctgca tagtattcca tggtgtatat |
| 5281 | gtgccacatt ttcttaatcc agtctatcat tgttggacat ttgggttggt tccaagtctt |
| 5341 | tgctattgtg aatagtgctg caataaacat atgtgtgcat gtgtctttaa aaaaaaaaa |

The polypeptide sequence of human FGF5 (transcript variant 2) is depicted in SEQ ID NO: 3. The nucleotide sequence of human FGF5 (transcript variant 2) is shown in SEQ ID NO: 4. Sequence information related to FGF5 (transcript variant 2) is accessible in public databases by GenBank Accession numbers NP_{—}149134 (protein) and NM_{—}033143 (nucleic acid). The polypeptide sequence is also depicted in FIG. 2.

SEQ ID NO: 3 is the human wild type amino acid sequence corresponding to FGF5 isoform 2 (residues 1-123):

| | |
|---|---|
| 1 | MSLSFLLLLF FSHLILSAWA HGEKRLAPKG QPGPAATDRN |
| | PRGSSSRQSS SSAMSSSSAS |
| 61 | SSPAASLGSQ GSGLEQSSFQ WSPSGRRTGS LYCRVGIGFH |
| | LQIYPDGKVN GSHEANMLSQ |
| 121 | VHR |

SEQ ID NO: 4 is the human wild type nucleotide sequence corresponding to FGF5 (transcript variant 2) (nucleotides 1-5295), wherein the underscored bolded “ATG” denotes the beginning of the open reading frame:

| | |
|---|---|
| 1 | ggggaagctt cgcaggcgtg cacggagcag tgagatcact ggcgttataa atatcccggt |
| 61 | gccagcgcgg agatccgctc gggtggcctc tctcttcccc tctccccttc tcttccccga |
| 121 | ggctatgtcc acccggtgcg gcgaggcggg cagagccaga ggcacgcagc cgcacagggg |
| 181 | ctacagagcc cagaatcagc cctacaagat gcacttagga cccccgcggc tggaagaatg |
| 241 | agcttgtcct tcctcctcct cctcttcttc agccacctga tcctcagcgc ctgggctcac |
| 301 | ggggagaagc gtctcgcccc caaagggcaa cccggacccg ctgccactga taggaaccct |
| 361 | agaggctcca gcagcagaca gagcagcagt agcgctatgt cttcctcttc tgcctcctcc |
| 421 | tcccccgcag cttctctggg cagccaagga agtggcttgg agcagagcag tttccagtgg |
| 481 | agcccctcgg ggcgccggac cggcagcctc tactgcagag tgggcatcgg tttccatctg |
| 541 | cagatctacc cggatggcaa agtcaatgga tcccacgaag ccaatatgtt aagccaagtt |
| 601 | cacagatgac tgcaagttca gggagcgttt tcaagaaaat agctataata cctatgcctc |
| 661 | agcaatacat agaactgaaa aaacagggcg ggagtggtat gtggccctga ataaaagagg |
| 721 | aaaagccaaa cgagggtgca gcccccgggt taaaccccag catatctcta cccattttct |
| 781 | gccaagattc aagcagtcgg agcagccaga actttctttc acggttactg ttcctgaaaa |
| 841 | gaaaaagcca cctagcccta tcaagccaaa gattcccctt tctgcacctc ggaaaaatac |
| 901 | caactcagtg aaatacagac tcaagtttcg ctttggataa tattcctctt ggccttgtga |
| 961 | gaaaccattc tttcccctca ggagtttcta taggtgtctt cagagttctg aagaaaaatt |
| 1021 | actggacaca gcttcagcta tacttacact gtattgaagt cacgtcattt gtttcaatgt |
| 1081 | gactgaaaca aaatgttttt tgataggaag gaaactggaa ttctttgtac taatacaggg |
| 1141 | agcacactcc ttcagttcag caagacataa agccttttgc tttatgcttg agggatattt |
| 1201 | agaactttgt attttcggaa agttaaataa cagggactac gtatttttct gacttttaca |
| 1261 | gattaacctg aaagaacata catgatacat ttttattttt ggtttccaaa gaatattttg |
| 1321 | atgcagataa aatattttgt taacttttgt ttttttttgt ttgttttctt aaaagtacct |
| 1381 | ctgcattgag catattttct tacttttatt attttaatta atatgacata agcaatcatt |
| 1441 | ttatgctgtt tatgaattat aaatgtgttt atagctcatt tgtaatatgg aaatctttta |
| 1501 | catttttcct attcactgca cttttttatt gtttttattt ctagccatac ctcagataat |
| 1561 | atgtttagtt ttacatttta aaatgtttaa attctctttc acagcaccaa aggctcagct |
| 1621 | tggatttgtg tgtatgtgta tgtcaattca tgacattatg tggaatccta aacctttggt |
| 1681 | ggctgggata tgatgggtta gaagcaagga gaaaatataa ggactttttg atggaattaa |
| 1741 | atgtgggagg taaggaaaag gatttagagg taaaagtaca ctaagtttgc aacatttatt |
| 1801 | gagatctaag tctgtcttgc cttcatttct ctttttatct cccccttgcc ctcattcttg |
| 1861 | aacagctgga ggaatacatt ttattctgtc catgaagcat acactatgaa attcaagtgc |
| 1921 | ttaaaaatac ttctatgact ctctgctatc ccactgtata gatccacagg gagcaaacac |
| 1981 | ttagaaatga tagagaactg aaggagatca atggtttaac agttatccat gccaagtccc |
| 2041 | attgtcagaa atattcttat tactcagtca aacactcttt gagcttccct tcctaaaggt |
| 2101 | aaccaatcca gtgaatagat gtgccctttt ataaggaaac ttctgatgtt tattaaaaaa |
| 2161 | actggccttt tgatagaggt aacttaattt gggaatttgt tgtgttgaaa tggcatttaa |
| 2221 | tttcaaccta aatactgact gctggacata aatcacagaa aatttaactt aagaaaattt |
| 2281 | acaaaattta ttctcaggta atcattttaa taaagttctg caaaatacac gtttatctta |
| 2341 | cattcagaaa tgtggcaaaa aaggcatagc taaaggctaa acatatggct ttagtagtaa |
| 2401 | caaaagggtt catagaaact tcatggtttg catttaaaca tgtttaaagt gtacttataa |
| 2461 | actatttttt tcttaaagca aactatgatt tattttggtg cacaaataca aagtggaaac |
| 2521 | ttaccaaaat tgaactagct accatataag cagattgctt taatttgatg ggaaaatagt |
| 2581 | acacacatat atataacaaa taatatatta aaaaacccat ccatcaacta aaacattata |
| 2641 | tgtatacatc agtatagtgt tttattataa agccaattat ctgattaagc attctttcca |
| 2701 | ctgaatgcat aatgtttaaa tagcataaaa tgaaatgcta caaaaattga actaatttat |
| 2761 | actttaaagt atttctgggt taaatgaaac aatgaaattt tttagtatgt tcaactctca |
| 2821 | tccaaatggc atatgaccct gtttacacag cctaaagcta aaaatattac tctagtttat |
| 2881 | tctaatctat tgttaagtat tgtgcactgt ataccaagtt cttagggcac atgaaaaatt |
| 2941 | ttagctgcca aacaggaact agtaaacata tgttcctaat aagtgaaggg aaagataata |
| 3001 | atgatggtca acaataagcc acgtcaatgc ataagttgta taggctaaat gttgcttgta |
| 3061 | ggctacatta aactcaaatg taatagttta tcttatactc ctggtttgat ttgattagca |
| 3121 | tattaacgtg aaagtaggat agctactaaa tatatattat gcaagtcagg aatcattaat |
| 3181 | ttcaaaattt aaagccatgc taaaattaaa aagaaaatat taaattacac aattacactt |
| 3241 | gtctttactg gccatacaaa atgatttttt tttttttttt gagacagagt cttgctctgt |
| 3301 | caccaggctg gagtgcagtg gcatgatctc ggctcactgc aacctccaac tccctggttt |
| 3361 | aagggattct cctgcctcag cctcccaagt agctgggatt acagactcat gccaccacgc |
| 3421 | cagctaattt ttgtattttt agtagagacg gggtttcacc atgttggtca ggatggtctc |
| 3481 | aatcctggcc tcttgatagt cctgacctca tgatctgccc acctcggcct ccccaaagtg |
| 3541 | ctgggattac aggtacaatg atgtataatt aatgcttagt gaagcataaa gttacctaca |
| 3601 | tcaattaatt aaatgaactt atgtacagaa aacatgtata aatataagtc tatactaatg |
| 3661 | cttacaactt tctaagaggg ttcttgctta tgtagctttt tattatttta agtaactaga |
| 3721 | accaccaaat atcaaataaa attatttggt tatggttatg ttcatctaaa cacaacaata |
| 3781 | acttttatat taatatttag gagtctattt tgtctatagg tgacaaacat ctccagacta |
| 3841 | acatgtcagt tttatcaatt atattatgtt taattattta agatttcttt atgtggaaca |
| 3901 | tctatagaga taaatagaaa ttttcaataa gatgtagtaa cactgtgatt tatctttcaa |
| 3961 | gagtctctct tcacttcctt ctaaagagac taatttgaga gtacaggtgc atattaattt |
| 4021 | tcttggttct ttcagctgaa ttatattggt ccagaagttc aaaatcatgt gacaataata |
| 4081 | agggatactg acagaagtta tttccaagtt tgtgtatata ttataaaaat tacatatata |
| 4141 | aaactaaggc ttttatttct gttattttta agcttttatt tcttgtagct aaaaataaaa |
| 4201 | catcataaat ctggtaggta aatttcttat taaatcaatc ttgaaataga aaatgtaata |
| 4261 | actttcttac cattaacatt ttttaccctt ccatagaagg gagggaataa atcatgactt |
| 4321 | atcccatttt caataacaaa acgaaactat ggcactaacc aaaaacttgc attctggcat |
| 4381 | aatttttaca gttgcagaga attgtttctg ggctcattaa aaaaagtagt attgcagaca |
| 4441 | ttgctgcaat gggaagcaga caataacttc ttaaaggaat tctacacctc ctttaagatt |
| 4501 | tacttaattg ctacatctaa attctgataa tttaaaatcc attttaggtg ataaaatttt |
| 4561 | ttaaaagttt tgaaggaaac ctctggataa atggacaagg cctaattttt ttttgtagtc |
| 4621 | aatccaactg tactggccaa tttttgaaat aagattatat gattaggtat tagcagagac |
| 4681 | aaagagttac ctcctccatc ttactctgcc ctatttgaaa gtctcagggg agaaaaggga |
| 4741 | acaagatgct gatccaacct gagtggagtc aggtgaggca tctttacatc taagaatttt |
| 4801 | tttttaaatt ttattattat tatacttcaa gttctagggt acatgtccac aatgcacatg |
| 4861 | tctgtcacac atgcacacat gtgccatgct ggtgtgctgc acccaccaac ctgtcatcca |
| 4921 | gcattaggta tatctcctaa tgctatccct cccctctcca cccaccccac agcaggcccc |
| 4981 | ggtatgtgat gttccccttc gtgtgtccat gtgttcttat tgttcaattc ccacctatga |
| 5041 | gtgagaatat gtggtgtttg gtttttggtc cttgcaatag tttgctgaga atgatggttt |
| 5101 | ccagcttcat ccatgtccct acaaagaaca tgaactcatc attttttatg gctgcatagt |
| 5161 | attccatggt gtatatgtgc cacattttct taatccagtc tatcattgtt ggacatttgg |
| 5221 | gttggttcca agtctttgct attgtgaata gtgctgcaat aaacatatgt gtgcatgtgt |
| 5281 | ctttaaaaaa aaaaa |

As used herein, a “FGF5 molecule” can be a nucleic acid which encodes a polypeptide that exhibits FGF5 activity, or a polypeptide or peptidomimetic that exhibits FGF5 activity. For example, a FGF5 molecule can include the human FGF5 protein (e.g., having the amino acid sequence shown in SEQ ID NO: 1 or 3), or a variant thereof, such as a fragment thereof, that exhibits FGF5 activity. FGF5 activity can encompass signaling events by way of its receptor, the FGFR1 receptor. For example, FGF5 activity can be a signal transduced in response to downstream signaling by binding to the FGFR1 receptor and inducing tyrosine phosphorylation events.

Protein variants can include amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture.

Nucleic acid sequences comprising a gene, such as a FGF5 gene, that encodes a polypeptide can be synthesized, in whole or in part, using chemical methods known in the art. Alternatively, a polypeptide, such as FGF5, can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of FGF5 polypeptides can be separately synthesized and combined using chemical methods to produce a full-length molecule.

The nucleic acid can be any type of nucleic acid, including genomic DNA, complementary DNA (cDNA), synthetic or semi-synthetic DNA, as well as any form of corresponding RNA. For example, a FGF5 molecule can comprise a recombinant nucleic acid encoding human FGF5 protein. In one embodiment, a FGF5 molecule can comprise a non-naturally occurring nucleic acid created artificially (such as by assembling, cutting, ligating or amplifying sequences). A FGF5 molecule can be double-stranded. A FGF5 molecule can be single-stranded. The FGF5 molecules of the presently disclosed subject matter can be obtained from various sources and can be produced according to various techniques known in the art. For example, a nucleic acid that is a FGF5 molecule can be obtained by screening DNA libraries, or by amplification from a natural source. The FGF5 molecules can be produced via recombinant DNA technology and such recombinant nucleic acids can be prepared by conventional techniques, including chemical synthesis, genetic engineering, enzymatic techniques, or a combination thereof. Non-limiting examples of a FGF5 molecule that is a nucleic acid, is the nucleic acid comprising SEQ ID NO: 2 or 4. Another example of a FGF5 molecule is a fragment of a nucleic acid comprising the sequence shown in SEQ ID NO: 2 or 4, wherein the fragment exhibits FGF5 activity. A FGF5 molecule of this presently disclosed subject matter also encompasses variants of the human nucleic acid encoding the FGF5 protein, or variants of the human FGF5 proteins that exhibit FGF5 activity. A FGF5 molecule can also include a fragment of the human FGF5 nucleic acid which encodes a polypeptide that exhibits FGF5 activity. A FGF5 molecule can encompass a fragment of the human FGF5 protein that exhibits FGF5 activity.

A FGF5 molecule can also encompass FGF5 ortholog genes, which are genes conserved among different biological species such as humans, dogs, cats, mice, and rats, that encode proteins (for example, homologs (including splice variants), mutants, and derivatives) having biologically equivalent functions as the human-derived protein (such as a FGF5 protein). FGF5 orthologs include any mammalian ortholog of FGF5 inclusive of the ortholog in humans and other primates, experimental mammals (such as mice, rats, hamsters and guinea pigs), mammals of commercial significance (such as horses, cows, camels, pigs and sheep), and also companion mammals (such as domestic animals, e.g., rabbits, ferrets, dogs, and cats).

The FGF5 variants can comprise, for instance, naturally-occurring variants due to allelic variations between individuals (e.g., polymorphisms), mutated alleles related to alopecia areata, or alternative splicing forms. In one embodiment, a FGF5 molecule is a nucleic acid variant of the nucleic acid having the sequence shown in SEQ ID NO: 2 or 4, wherein the variant has a nucleotide sequence identity to SEQ ID NO: 2 or 4 of at least about 65%, at least about 75%, at least about 85%, at least about 90%, at least about 91%, at least about 92%, at least about 93%, at least about 94%, at least about 95%, at least about 96%, at least about 97%, at least about 98%, or at least about 99% with SEQ ID NO: 2 or 4. In one embodiment, a FGF5 molecule encompasses any portion of at least about 8 consecutive nucleotides of SEQ ID NO: 2 or 4. In one embodiment, the fragment can comprise at least about 15 nucleotides, at least about 20 nucleotides, or at least about 30 nucleotides of SEQ ID NO: 2 or 4. Fragments include all possible nucleotide lengths between about 8 and 100 nucleotides, for example, lengths between about 15 and 100, or between about 20 and 100.

The presently disclosed subject matter further provides for nucleic acids that are complementary to a nucleic acid encoding a FGF5 protein. Such complementary nucleic acids can comprise nucleic acid sequences, which hybridize to a nucleic acid sequence encoding a FGF5 protein under stringent hybridization conditions. Non-limiting examples of stringent hybridization conditions include temperatures above 30° C., above 35° C., in excess of 42° C., and/or salinity of less than about 500 mM, or less than 200 mM. Hybridization conditions can be adjusted by the skilled artisan via modifying the temperature, salinity and/or the concentration of other reagents such as SDS or SSC.

In one embodiment, a FGF5 molecule comprises a protein or polypeptide encoded by a FGF5 nucleic acid sequence, such as the sequence shown in SEQ ID NO: 1 or 3. In another embodiment, the polypeptide can be modified, such as by glycosylations and/or acetylations and/or chemical reaction or coupling, and can contain one or several non-natural or synthetic amino acids. An example of a FGF5 molecule is the polypeptide having the amino acid sequence shown in SEQ ID NO: 1 or 3. In another embodiment, a FGF5 molecule can be a fragment of a FGF5 protein. For example, the FGF5 molecule can encompass any portion of at least about 8 consecutive amino acids of SEQ ID NO: 1 or 3. The fragment can comprise at least about 10 amino acids, a least about 20 amino acids, at least about 30 amino acids, at least about 40 amino acids, a least about 50 amino acids, at least about 60 amino acids, or at least about 75 amino acids of SEQ ID NO: 1 or 3. Fragments include all possible amino acid lengths between about 8 and 100 about amino acids, for example, lengths between about 10 and 100 amino acids, between about 15 and 100 amino acids, between about 20 and 100 amino acids, between about 35 and 100 amino acids, between about 40 and 100 amino acids, between about 50 and 100 amino acids, between about 70 and 100 amino acids, between about 75 and 100 amino acids, or between about 80 and 100 amino acids.

In certain embodiments, the FGF5 molecule includes variants of the human FGF5 protein (comprising the amino acid sequence shown in SEQ ID NO: 1 or 3). Such variants can include those having at least from about 46% to about 50% identity to SEQ ID NO: 1 or 3, or having at least from about 50.1% to about 55% identity to SEQ ID NO: 1 or 3, or having at least from about 55.1% to about 60% identity to SEQ ID NO: 1 or 3, or having from at least about 60.1% to about 65% identity to SEQ ID NO: 1 or 3, or having from about 65.1% to about 70% identity to SEQ ID NO: 1 or 3, or having at least from about 70.1% to about 75% identity to SEQ ID NO: 1 or 3, or having at least from about 75.1% to about 80% identity to SEQ ID NO: 1 or 3, or having at least from about 80.1% to about 85% identity to SEQ ID NO: 1 or 3, or having at least from about 85.1% to about 90% identity to SEQ ID NO: 1 or 3, or having at least from about 90.1% to about 95% identity to SEQ ID NO: 1 or 3, or having at least from about 95.1% to about 97% identity to SEQ ID NO: 1 or 3, or having at least from about 97.1% to about 99% identity to SEQ ID NO: 1 or 3.

Techniques for making substitution mutations at predetermined sites in DNA having a known sequence are well known, for example M13 primer mutagenesis and PCR mutagenesis. Amino acid substitutions can be single residues, but can occur at a number of different locations at once. In one non-limiting embodiment, insertions can be on the order of about from 1 to about 10 amino acid residues, while deletions can range from about 1 to about 30 residues. Deletions or insertions can be made in adjacent pairs (for example, a deletion of about 2 residues or insertion of about 2 residues). Substitutions, deletions, insertions, or any combination thereof can be combined to arrive at a final construct. The mutations cannot place the sequence out of reading frame and should not create complementary regions that can produce secondary mRNA structure. Substitutional variants are those in which at least one residue has been removed and a different residue inserted in its place.

Substantial changes in function or immunological identity are made by selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions that can produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Minor variations in the amino acid sequences of proteins are provided by the presently disclosed subject matter. The variations in the amino acid sequence can be when the sequence maintains at least about 30%, at least about 40%, at least about 50%, at least about 60%, at least about 70%, at least about 75%, at least about 80%, at least about 90%, at least about 95%, or at least about 99% identity to SEQ ID NO: 1 or 3. For example, conservative amino acid replacements can be utilized. Conservative replacements are those that take place within a family of amino acids that are related in their side chains, wherein the interchangeability of residues have similar side chains.

Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) a group of amino acids having aliphatic-hydroxyl side chains, such as serine and threonine; (ii) a group of amino acids having amide-containing side chains, such as asparagine and glutamine; (iii) a group of amino acids having aliphatic side chains such as glycine, alanine, valine, leucine, and isoleucine; (iv) a group of amino acids having aromatic side chains, such as phenylalanine, tyrosine, and tryptophan; and (v) a group of amino acids having sulfur-containing side chains, such as cysteine and methionine. Useful conservative amino acids substitution groups are: valine-leucine-isoleucine, phenylalanine-tyrosine, lysine-arginine, alanine valine, glutamic-aspartic, and asparagine-glutamine.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

In another embodiment, the FGF5 molecule encompasses a peptidomimetic which exhibits FGF5 activity. A peptidomimetic is a small protein-like chain designed to mimic a peptide that can arise from modification of an existing peptide in order to protect that molecule from enzyme degradation and increase its stability, and/or alter the molecule's properties (e.g., modifications that change the molecule's stability or biological activity). These modifications involve changes to the peptide that cannot occur naturally (such as altered backbones and the incorporation of non-natural amino acids). Drug-like compounds can be developed from existing peptides. A peptidomimetic can be a peptide, partial peptide, or non-peptide molecule that mimics the tertiary binding structure or activity of a selected native peptide or protein functional domain (e.g., binding motif or active site). These peptide mimetics include recombinantly or chemically modified peptides.

In one embodiment, a FGF5 molecule comprising SEQ ID NO: 1 or 3, variants of such, or fragments thereof, can be modified to produce peptide mimetics by replacement of one or more naturally occurring side chains of the 20 genetically encoded amino acids (or D amino acids) with other side chains. This can occur, for instance, with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, ifuryl, imidazolidinyl imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics can also have amino acid residues that have been chemically modified by phosphorylation, sulfonation, biotinylation, or the addition or removal of other moieties. For example, peptidomimetics can be designed and directed to amino acid sequences encoded by a FGF5 molecule comprising SEQ ID NO: 1 or 3.

A variety of techniques are available for constructing peptide mimetics with the same or similar desired biological activity as the corresponding native but with more favorable activity than the peptide with respect to solubility, stability, and/or susceptibility to hydrolysis or proteolysis (see, e.g., Morgan & Gainor, Ann. Rep. Med. Chem. 24,243-252, 1989). Certain peptidomimetic compounds are based upon the amino acid sequence of the peptides of the presently disclosed subject matter. Peptidomimetic compounds can be synthetic compounds having a three-dimensional structure (i.e. a peptide motif) based upon the three-dimensional structure of a selected peptide. The peptide motif provides the peptidomimetic compound with the desired biological activity, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life. Peptidomimetic design strategies are readily available in the art (see, e.g., Ripka & Rich (1998) Curr. Op. Chem. Biol. 2:441-452; Hruby et al. (1997) Curr. Op. Chem. Biol. 1:114-119; Hruby & Balse, (2000) Curr. Med. Chem. 9:945-970).

Bacterial and Yeast Expression Systems.

In bacterial systems, a number of expression vectors can be selected. For example, when a large quantity of a protein encoded by a gene, such as FGF5, is needed for the induction of antibodies, vectors which direct high level expression of proteins that are readily purified can be used. Non-limiting examples of such vectors include multifunctional E. coli cloning and expression vectors such as BLUESCRIPT (Stratagene). pIN vectors or pGEX vectors (Promega, Madison, Wis.) also can be used to express foreign polypeptide molecules as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

Plant and Insect Expression Systems.

If plant expression vectors are used, the expression of sequences encoding a FGF5 protein can be driven by any of a number of promoters. For example, viral promoters such as the 35S and 19S promoters of CaMV can be used alone or in combination with the omega leader sequence from TMV. Alternatively, plant promoters such as the small subunit of RUBISCO or heat shock promoters, can be used. These constructs can be introduced into plant cells by direct DNA transformation or by pathogen-mediated transfection.

An insect system also can be used to express the FGF5 protein. For example, in one such system Autographa californica nuclear polyhedrosis virus (AcNPV) is used as a vector to express foreign genes in Spodoptera frugiperda cells or in Trichoplusia larvae. Sequences encoding a polypeptide of FGF5 can be cloned into a non-essential region of the virus, such as the polyhedrin gene, and placed under control of the polyhedrin promoter. Successful insertion of nucleic acid sequences, such as a sequence corresponding to a gene, such as a FGF5 gene, will render the polyhedrin gene inactive and produce recombinant virus lacking coat protein. The recombinant viruses can then be used to infect S. frugiperda cells or Trichoplusia larvae in which the protein or a variant thereof can be expressed.

Mammalian Expression Systems.

An expression vector can include a nucleotide sequence that encodes a FGF5 polypeptide linked to at least one regulatory sequence in a manner allowing expression of the nucleotide sequence in a host cell. A number of viral-based expression systems can be used to express a FGF5 protein or a variant thereof in mammalian host cells. For example, if an adenovirus is used as an expression vector, sequences encoding a protein can be ligated into an adenovirus transcription/translation complex comprising the late promoter and tripartite leader sequence. Insertion into a non-essential E1 or E3 region of the viral genome can be used to obtain a viable virus which expresses a FGF5 protein in infected host cells. Transcription enhancers, such as the Rous sarcoma virus (RSV) enhancer, can also be used to increase expression in mammalian host cells.

Regulatory sequences are well known in the art, and can be selected to direct the expression of a protein or polypeptide of interest in an appropriate host cell as described in Goeddel, Gene Expression Technology: Methods in Enzymology 185, Academic Press, San Diego, Calif. (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L, et al.,Curr Opin Genet Dev, 2002, 12(2):137-41) enhancers, and other expression control elements.

Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest is stably integrated into the genome of eukaryotic cells (for example mammalian cells, such as cells from the end bulb of the hair follicle), resulting in the stable expression of transfected genes. An exogenous nucleic acid sequence can be introduced into a cell (such as a mammalian cell, either a primary or secondary cell) by homologous recombination as disclosed in U.S. Pat. No. 5,641,670, the contents of which are herein incorporated by reference.

A gene that encodes a selectable marker (for example, resistance to antibiotics or drugs, such as ampicillin, neomycin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule (for example, a protein encoded by a gene, such as FGF5).

Cell Transfection

A eukaryotic expression vector can be used to transfect cells in order to produce proteins encoded by nucleotide sequences of the vector. Mammalian cells (such as isolated cells from the hair bulb; for example dermal sheath cells and dermal papilla cells) can contain an expression vector (for example, one that contains a gene encoding a FGF5 protein or polypeptide) via introducing the expression vector into an appropriate host cell via methods known in the art.

A host cell strain can be chosen for its ability to modulate the expression of the inserted sequences or to process the expressed polypeptide encoded by a gene, such as a FGF5 gene, in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextran-mediated transfection, or electroporation. Electroporation is carried out at approximate voltage and capacitance to result in entry of the DNA construct(s) into cells of interest (such as cells of the end bulb of a hair follicle, for example dermal papilla cells or dermal sheath cells). Other transfection methods also include modified calcium phosphate precipitation, polybrene precipitation, liposome fusion, and receptor-mediated gene delivery.

Cells that will be genetically engineered can be primary and secondary cells obtained from various tissues, and include cell types which can be maintained and propagated in culture. Non-limiting examples of primary and secondary cells include epithelial cells (for example, dermal papilla cells, hair follicle cells, inner root sheath cells, outer root sheath cells, sebaceous gland cells, epidermal matrix cells), neural cells, endothelial cells, glial cells, fibroblasts, muscle cells (such as myoblasts) keratinocytes, formed elements of the blood (e.g., lymphocytes, bone marrow cells), and precursors of these somatic cell types.

Vertebrate tissue can be obtained by methods known to one skilled in the art, such a punch biopsy or other surgical methods of obtaining a tissue source of the primary cell type of interest. In one embodiment, a punch biopsy or removal can be used to obtain a source of keratinocytes, fibroblasts, endothelial cells, or mesenchymal cells (for example, hair follicle cells or dermal papilla cells). In another embodiment, removal of a hair follicle can be used to obtain a source of fibroblasts, keratinocytes, endothelial cells, or mesenchymal cells (for example, hair follicle cells or dermal papilla cells). A mixture of primary cells can be obtained from the tissue, using methods readily practiced in the art, such as explanting or enzymatic digestion (for examples using enzymes such as pronase, trypsin, collagenase, elastase dispase, and chymotrypsin). Biopsy methods have also been described in U.S. Pat. No. 7,419,661 and PCT application publication WO/2001/032840, and are hereby each incorporated by reference in their entireties.

Primary cells can be acquired from the individual to whom the genetically engineered primary or secondary cells are administered. However, primary cells can also be obtained from a donor, other than the recipient, of the same species. The cells can also be obtained from another species (for example, rabbit, cat, mouse, rat, sheep, goat, dog, horse, cow, bird, or pig). Primary cells can also include cells from an isolated vertebrate tissue source grown attached to a tissue culture substrate (for example, flask or dish) or grown in a suspension; cells present in an explant derived from tissue; both of the aforementioned cell types plated for the first time; and cell culture suspensions derived from these plated cells. Secondary cells can be plated primary cells that are removed from the culture substrate and replated, or passaged, in addition to cells from the subsequent passages. Secondary cells can be passaged one or more times. These primary or secondary cells can contain expression vectors having a gene that encodes a protein of interest (for example, a FGF5 protein or polypeptide).

Cell Culturing

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized. Non-limiting examples of medium include, for example, Minimal Essential Medium (MEM, Sigma, St. Louis, Mo.); Dulbecco's Modified Eagles Medium (DMEM, Sigma); Ham's F10 Medium (Sigma); HyClone cell culture medium (HyClone, Logan, Utah); RPMI-1640 Medium (Sigma); and chemically-defined (CD) media, which are formulated for various cell types, e.g., CD-CHO Medium (Invitrogen, Carlsbad, Calif.).

The cell culture media can be supplemented as necessary with supplementary components or ingredients, including optional components, in appropriate concentrations or amounts, as necessary or desired. Cell culture medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that can be required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, and epidermal growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example pluronic polyol; and (8) galactose. In one embodiment, soluble factors can be added to the culturing medium.

The mammalian cell culture that can be used with the presently disclosed subject matter is prepared in a medium suitable for the type of cell being cultured. In one embodiment, the cell culture medium can be any one of those previously discussed (for example, MEM) that is supplemented with serum from a mammalian source (for example, fetal bovine serum (FBS)). In another embodiment, the medium can be a conditioned medium to sustain the growth of epithelial cells or cells obtained from the hair bulb of a hair follicle (such as dermal papilla cells or dermal sheath cells). For example, epithelial cells can be cultured according to Barnes and Mather in Animal Cell Culture Methods (Academic Press, 1998), which is hereby incorporated by reference in its entirety. In a further embodiment, epithelial cells or hair follicle cells can be transfected with DNA vectors containing genes that encode a polypeptide or protein of interest (for example, a FGF5 protein or polypeptide). In other embodiments of the presently disclosed subject matter, cells are grown in a suspension culture (for example, a three-dimensional culture such as a hanging drop culture) in the presence of an effective amount of enzyme, wherein the enzyme substrate is an extracellular matrix molecule in the suspension culture. For example, the enzyme can be a hyaluronidase. Epithelial cells or hair follicle cells can be cultivated according to methods practiced in the art, for example, as those described in U.S. Pat. No. 7,785,876, or as described by Harris in Handbook in Practical Animal Cell Biology: Epithelial Cell Culture (Cambridge Univ. Press, Great Britain; 1996; see Chapter 8), which are each hereby incorporated by reference.

A suspension culture is a type of culture wherein cells, or aggregates of cells (such as aggregates of DP cells), multiply while suspended in liquid medium. A suspension culture comprising mammalian cells can be used for the maintenance of cell types that do not adhere or to enable cells to manifest specific cellular characteristics that are not seen in the adherent form. Some types of suspension cultures can include three-dimensional cultures or a hanging drop culture. A hanging-drop culture is a culture in which the material to be cultivated is inoculated into a drop of fluid attached to a flat surface (such as a coverglass, glass slide, Petri dish, flask, and the like), and can be inverted over a hollow surface. Cells in a hanging drop can aggregate toward the hanging center of a drop as a result of gravity. However, according to the methods of the presently disclosed subject matter, cells cultured in the presence of a protein that degrades the extracellular matrix (such as collagenase, chondroitinase, hyaluronidase, and the like) will become more compact and aggregated within the hanging drop culture, for degradation of the ECM will allow cells to become closer in proximity to one another since less of the ECM will be present. See also U.S. Patent Publication No. US 2010-0303767 A1, which is incorporated by reference.

Cells obtained from the hair bulb of a hair follicle (such as dermal papilla cells or dermal sheath cells) can be cultured as a single, homogenous population (for example, comprising DP cells) in a hanging drop culture so as to generate an aggregate of DP cells. Cells can also be cultured as a heterogeneous population (for example, comprising DP and DS cells) in a hanging drop culture so as to generate a chimeric aggregate of DP and DS cells. Epithelial cells can be cultured as a monolayer to confluency as practiced in the art. Such culturing methods can be carried out essentially according to methods described in Chapter 8 of the Handbook in Practical Animal Cell Biology: Epithelial Cell Culture (Cambridge Univ. Press, Great Britain; 1996); Underhill C B, J Invest Dermatol, 1993, 101(6):820-6); in Armstrong and Armstrong, (1990) J Cell Biol 110:1439-55; or in Animal Cell Culture Methods (Academic Press, 1998), which are all hereby incorporated by reference in their entireties.

Three-dimensional cultures can be formed from agar (such as Gey's Agar), hydrogels (such as matrigel, agarose, and the like; Lee et al., (2004) Biomaterials 25: 2461-2466) or polymers that are cross-linked. These polymers can comprise natural polymers and their derivatives, synthetic polymers and their derivatives, or a combination thereof. Natural polymers can be anionic polymers, cationic polymers, amphipathic polymers, or neutral polymers. Non-limiting examples of anionic polymers can include hyaluronic acid, alginic acid (alginate), carageenan, chondroitin sulfate, dextran sulfate, and pectin. Some examples of cationic polymers, include but are not limited to, chitosan or polylysine. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73). Examples of amphipathic polymers can include, but are not limited to collagen, gelatin, fibrin, and carboxymethyl chitin. Non-limiting examples of neutral polymers can include dextran, agarose, or pullulan. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73).

Cells suitable for culturing according to methods of the presently disclosed subject matter can harbor introduced expression vectors, such as plasmids. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 2001, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

Obtaining and Purifying Polypeptides

A polypeptide molecule encoded by a gene, such as a FGF5 gene, or a variant thereof, can be obtained by purification from human cells expressing a protein or polypeptide encoded by a FGF5 gene via in vitro or in vivo expression of a nucleic acid sequence encoding a FGF5 protein or polypeptide; or by direct chemical synthesis.

Detecting Polypeptide Expression.

Host cells which contain a nucleic acid encoding a FGF5 protein or polypeptide, and which subsequently express a protein encoded by a FGF5 gene, can be identified by various procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a nucleic acid encoding a FGF5 protein or polypeptide can be detected by DNA-DNA or DNA-RNA hybridization or amplification using probes or fragments of nucleic acids encoding a FGF5 protein or polypeptide. In one embodiment, a fragment of a nucleic acid of a FGF5 gene can encompass any portion of at least about 8 consecutive nucleotides of SEQ ID NO: 2 or 4. In another embodiment, the fragment can comprise at least about 10 consecutive nucleotides, at least about 15 consecutive nucleotides, at least about 20 consecutive nucleotides, or at least about 30 consecutive nucleotides of SEQ ID NO: 2 or 4. Fragments can include all possible nucleotide lengths between about 8 and about 100 nucleotides, for example, lengths between about 15 and about 100 nucleotides, or between about 20 and about 100 nucleotides. Nucleic acid amplification-based assays involve the use of oligonucleotides selected from sequences encoding a polypeptide encoded by a FGF5 gene to detect transformants which contain a nucleic acid encoding a FGF5 protein or polypeptide.

Protocols for detecting and measuring the expression of a polypeptide encoded by a gene, such as a FGF5 gene, using either polyclonal or monoclonal antibodies specific for the polypeptide are well established. Non-limiting examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a polypeptide encoded by a gene, such as a FGF5 gene, can be used, or a competitive binding assay can be employed.

Labeling and conjugation techniques are known by those skilled in the art and can be used in various nucleic acid and amino acid assays. Methods for producing labeled hybridization or PCR probes for detecting sequences related to nucleic acid sequences encoding a protein, such as FGF5, include, but are not limited to, oligolabeling, nick translation, end-labeling, or PCR amplification using a labeled nucleotide. Alternatively, nucleic acid sequences encoding a polypeptide encoded by a gene, such as a FGF5 gene, can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, and/or magnetic particles.

Expression and Purification of Polypeptides.

Host cells transformed with a nucleic acid sequence encoding a polypeptide, such as FGF5, can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding a polypeptide, such as FGF5, can be designed to contain signal sequences which direct secretion of soluble polypeptide molecules encoded by a gene, such as a FGF5 gene, or a variant thereof, through a prokaryotic or eukaryotic cell membrane or which direct the membrane insertion of membrane-bound a polypeptide molecule encoded by a FGF5 gene or a variant thereof.

Other constructions can also be used to join a gene sequence encoding a FGF5 polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Including cleavable linker sequences (i.e., those specific for Factor Xa or enterokinase (Invitrogen, San Diego, Calif.)) between the purification domain and a polypeptide encoded by a FGF5 gene also can be used to facilitate purification. One such expression vector provides for expression of a fusion protein containing a polypeptide encoded by a FGF5 gene and 6 histidine residues preceding a thioredoxin or an enterokinase cleavage site. The histidine residues facilitate purification by immobilized metal ion affinity chromatography, while the enterokinase cleavage site provides a means for purifying the polypeptide encoded by a FGF5 gene.

A FGF5 polypeptide can be purified from any human or non-human cell which expresses the polypeptide, including those which have been transfected with expression constructs that express a FGF5 protein. A purified FGF5 protein can be separated from other compounds which normally associate with a protein encoded by a FGF5 gene in the cell, such as certain proteins, carbohydrates, or lipids, using methods practiced in the art. Non-limiting methods include size exclusion chromatography, ammonium sulfate fractionation, ion exchange chromatography, affinity chromatography, and preparative gel electrophoresis.

Chemical Synthesis.

Nucleic acid sequences comprising a gene, such as a FGF5 gene, that encodes a polypeptide can be synthesized, in whole or in part, using chemical methods known in the art. Alternatively, a polypeptide, such as FGF5, can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of FGF5 polypeptides can be separately synthesized and combined using chemical methods to produce a full-length molecule. In one embodiment, a fragment of a nucleic acid sequence that comprises a FGF5 gene can encompass any portion of at least about 8 consecutive nucleotides of SEQ ID NO: 2 or 4. In one embodiment, the fragment can comprise at least about 10 nucleotides, at least about 15 nucleotides, at least about 20 nucleotides, or at least about 30 nucleotides of SEQ ID NO: 2 or 4. Fragments include all possible nucleotide lengths between about 8 and about 100 nucleotides, for example, lengths between about 15 and about 100 nucleotides, or between about 20 and about 100 nucleotides.

A FGF5 fragment can be a fragment of a protein, such as FGF5. For example, the FGF5 fragment can encompass any portion of at least about 8 consecutive amino acids of SEQ ID NO: 1 or 3. The fragment can comprise at least about 10 consecutive amino acids, at least about 20 consecutive amino acids, at least about 30 consecutive amino acids, at least about 40 consecutive amino acids, a least about 50 consecutive amino acids, at least about 60 consecutive amino acids, at least about 70 consecutive amino acids, or at least about 75 consecutive amino acids of SEQ ID NO: 1 or 3. Fragments include all possible amino acid lengths between about 8 and 100 about amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids.

A synthetic peptide can be substantially purified via high performance liquid chromatography (HPLC). The composition of a synthetic polypeptide of FGF5 can be confirmed by amino acid analysis or sequencing. Additionally, any portion of an amino acid sequence comprising a protein encoded by a FGF5 gene can be altered during direct synthesis and/or combined using chemical methods with sequences from other proteins to produce a variant polypeptide or a fusion protein.

Identifying FGF5 Modulating Compounds

The presently disclosed subject matter provides methods for identifying compounds which can be used for controlling and/or regulating hair growth (for example, hair density) or hair pigmentation in a subject. Since the presently disclosed subject matter has provided the identification of the gene listed herein as a gene associated with a hair loss disorder, the presently disclosed subject matter also provides methods for identifiying compounds that modulate the expression or activity of a gene and/or protein of FGF5. In addition, the presently disclosed subject matter provides methods for identifying compounds which can be used for the treatment of a hair loss disorder. The presently disclosed subject matter also provides methods for identifying compounds which can be used for the treatment of hypotrichosis (for example, hereditary hypotrichosis simplex (HHS)). Non-limiting examples of hair loss disorders include: androgenetic alopecia, Alopecia areata, telogen effluvium, alopecia areata, alopecia totalis, and alopecia universalis. The presently disclosed subject matter also provides methods for identifying compounds which can be used for the treatment of a hair growth disorder. The presently disclosed subject matter also provides methods for identifying compounds which can be used for the treatment of hypertrichosis (for example, X-linked hypertrichosis). Non-limiting examples of hair growth disorders include X-linked hypertrichosis, generalized hypertrichosis terminalis with or without gingival hyperplasia, autosomal recessive hypertrichosis, Cantu syndrome, Ambras type hypertrichosis and trichomegaly (e.g., ciliary or autosomal recessive trichomegaly). The methods can comprise the identification of test compounds or agents (e.g., peptides (such as antibodies or fragments thereof), small molecules, nucleic acids (such as siRNA or antisense RNA), or other agents) that can bind to a polypeptide molecule encoded by a FGF5 gene and/or have a stimulatory or inhibitory effect on the biological activity of a protein encoded by a FGF5 gene or its expression, and subsequently determining whether these compounds can regulate hair growth in a subject or can have an effect on symptoms associated with the hair loss disorders or hair growth disorders in an in vivo assay (i.e., examining an increase or reduction in hair growth).

As used herein, a “FGF5 modulating compound” refers to a compound that interacts with a FGF5 gene or a FGF5 protein or polypeptide and modulates its activity and/or its expression. The compound can either increase the activity or expression of a protein encoded by a FGF5 gene. The compound can be a FGF5 agonist (e.g., a FGF5 activator). In embodiment, the FGF5 activator is a polypeptide comprising SEQ ID NO: 1 or 3, or a fragment thereof; or a peptidomimetic comprising SEQ ID NO: 1 or 3. Conversely, the compound can decrease the activity or expression of a protein encoded by a FGF5 gene. The compound can be a FGF5 agonist or a FGF5 antagonist (e.g., a FGF5 inhibitor). Some non-limiting examples of FGF5 modulating compounds include peptides (such as peptide fragments comprising a polypeptide encoded by a FGF5 gene, or antibodies or fragments thereof), small molecules, and nucleic acids (such as siRNA or antisense RNA specific for a nucleic acid comprising a FGF5 gene). Agonists of a FGF5 protein can be molecules which, when bound to a FGF5 protein, increase or prolong the activity of the FGF5 protein. FGF5 agonists include, but are not limited to, proteins, nucleic acids, small molecules, or any other molecule which activates a FGF5 protein. Antagonists of a FGF5 protein can be molecules which, when bound to a FGF5 protein decrease the amount or the duration of the activity of the FGF5 protein. Antagonists include proteins, nucleic acids, antibodies, small molecules, or any other molecule which decrease the activity of a FGF5 protein.

The term “modulate,” as it appears herein, refers to a change in the activity or expression of a gene or protein of FGF5. For example, modulation can cause an increase or a decrease in protein activity, binding characteristics, or any other biological, functional, or immunological properties of a FGF5 protein.

In one embodiment, a FGF5 modulating compound can be a peptide fragment of a FGF5 protein that binds to the protein. For example, the FGF5 polypeptide can encompass any portion of at least about 8 consecutive amino acids of SEQ ID NO: 1 or 3. The fragment can comprise at least about 10 consecutive amino acids, at least about 20 consecutive amino acids, at least about 30 consecutive amino acids, at least about 40 consecutive amino acids, at least about 50 consecutive amino acids, at least about 60 consecutive amino acids, or at least about 75 consecutive amino acids of SEQ ID NO: 1 or 3. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England). The FGF5 peptide fragments can be isolated from a natural source, genetically engineered, or chemically prepared. These methods are well known in the art.

A FGF5 modulating compound can be a protein, such as an antibody (monoclonal, polyclonal, humanized, chimeric, or fully human), or a binding fragment thereof, directed against a polypeptide encoded by a FGF5 gene. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (see Roland E. Kontermann and Stefan Dübel (editors), Antibody Engineering, Vol. I & II, (2010) 2^{nd} ed., Springer; Antony S. Dimitrov (editor), Therapeutic Antibodies: Methods and Protocols (Methods in Molecular Biology), (2009), Humana Press; Benny Lo (editor) Antibody Engineering: Methods and Protocols (Methods in Molecular Biology), (2004) Humana Press, each of which are hereby incorporated by reference in their entireties). For example, antibodies directed to FGF5 can be obtained commercially from Abcam, Santa Cruz Biotechnology, Abgent, R&D Systems, Novus Biologicals, etc. Human antibodies directed to either FGF5 (such as monoclonal, humanized, or chimeric antibodies) can be useful antibody therapeutics for use in humans. In one embodiment, an antibody or binding fragment thereof is directed against SEQ ID NO: 1 or 3.

Inhibition of RNA encoding a polypeptide encoded by a FGF5 gene can effectively modulate the expression of a FGF5 gene from which the RNA is transcribed. Inhibitors are selected from the group comprising: siRNA; interfering RNA or RNAi; dsRNA; RNA Polymerase III transcribed DNAs; ribozymes; and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a polypeptide encoded by a FGF5 gene can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59). Antisense nucleotide sequences include, but are not limited to: morpholinos, 2′-O-methyl polynucleotides, DNA, RNA and the like. In one embodiment, the human FGF5 antisense oligonucleotide comprises CATACCACTCCCGCCCTGTT (SEQ ID NO: 105), CATACCACTCCCGCCCTGTTT (SEQ ID NO: 106); TACCACTCCCGCCCTGTTT (SEQ ID NO: 107); ATACCACTCCCGCCCTGTTT (SEQ ID NO: 108); or ATACCACTCCCGCCCTGTT (SEQ ID NO: 109). In one embodiment, the mouse FGF5 antisense oligonucleotide comprises GCTTTCCCTCTCTTGTTCAG (SEQ ID NO: 110); GCTTTCCCTCTCTTGTTCA (SEQ ID NO: 111); GCTTTCCCTCTCTTGTTCAGG (SEQ ID NO: 112); GGCTTTCCCTCTCTTGTTCAG (SEQ ID NO: 113); or GGCTTTCCCTCTCTTGTTCA (SEQ ID NO: 114).

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area. See also, McMnaus and Sharp (2002) Nat Rev Genetics, 3:737-47, and Sen and Blau (2006) FASEB J., 20:1293-99, the entire disclosures of which are herein incorporated by reference.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribo-nucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector (for example, see U.S. Pat. No. 7,294,504 and U.S. Pat. No. 7,422,896, the entire disclosures of which are herein incorporated by reference). Exemplary methods for producing and testing dsRNA or siRNA molecules are described in U.S. Patent Application Publication No. 2002/0173478 to Gewirtz, U.S. Pat. No. 8,071,559 to Hannon et al., U.S. Pat. No. 7,674,895 to Reich et al., and in U.S. Pat. No. 7,148,342 to Tolentino et al., the entire disclosures of which each are hereby incorporated by reference.

In one embodiment, an siRNA directed to a human nucleic acid sequence comprising a FGF5 gene can be generated against SEQ ID NO: 2 or 4. In another embodiment, an siRNA directed to a human nucleic acid sequence comprising a FGF5 gene can comprise any one of the sequences listed in Table 1.

In another embodiment, the siRNA directed to FGF5 is listed in Table 1.

TABLE 1
siRNA SEQUENCES for human FGF5

| SEQ ID NO: | SEQUENCE |
|---|---|
| 5 | GGTATGTGGCCCTGAATAA |
| 6 | GCAGAGTGGGCATCGGTTT |
| 7 | CCACGAAGCCAATATGTTA |
| 8 | ATACCAACTCAGTGAAATA |
| 9 | CCTGAATAAAAGAGGAAAA |
| 10 | GGAAATATTTGCTGTGTCT |
| 11 | CAGCATATCTCTACCCATT |
| 12 | TAAAAGAGGAAAAGCCAAA |
| 13 | CAAGTTTCGCTTTGGATAA |
| 14 | CAGAGCAGCAGTAGCGCTA |
| 15 | AGATCTACCCGGATGGCAA |
| 16 | GCAATACATAGAACTGAAA |
| 17 | TGTAGGAATACGAGGAGTT |
| 18 | GCAAGTTCAGGGAGCGTTT |
| 19 | GTGCCAAGTTCACAGATGA |
| 20 | AGCAATACATAGAACTGAA |
| 21 | TCAGGGGATTGTAGGAATA |
| 22 | ACAGATGACTGCAAGTTCA |
| 23 | CTATAATACCTATGCCTCA |
| 24 | AAATACCAACTCAGTGAAA |
| 25 | TCAGTGAAATACAGACTCA |
| 26 | GGAAGTGGCTTGGAGCAGA |
| 27 | GATCTACCCGGATGGCAAA |
| 28 | GATTGTAGGAATACGAGGA |
| 29 | TACCTATGCCTCAGCAATA |
| 30 | GAAATACAGACTCAAGTTT |
| 31 | TCAAGTTTCGCTTTGGATA |
| 32 | GAGCAGAGCAGTTTCCAGT |
| 33 | AAATATTTGCTGTGTCTCA |
| 34 | TGGCAAAGTCAATGGATCC |
| 35 | AGTCAATGGATCCCACGAA |
| 36 | CACGAAGCCAATATGTTAA |
| 37 | AAGCCAATATGTTAAGTGT |
| 38 | GTATGTGGCCCTGAATAAA |
| 39 | GCCCTGAATAAAAGAGGAA |
| 40 | TGAAATACAGACTCAAGTT |
| 41 | AGGAAAACTCCATGCAAGT |
| 42 | TGGTATGTGGCCCTGAATA |
| 43 | GGCTTGGAGCAGAGCAGTT |
| 44 | GTAGGAATACGAGGAGTTT |
| 45 | CCTCAGCAATACATAGAAC |
| 46 | AATAAAAGAGGAAAAGCCA |
| 47 | ATAAAAGAGGAAAAGCCAA |
| 48 | ACTCAGTGAAATACAGACT |
| 49 | GAGCAGCAGTAGCGCTATG |
| 50 | ATGGCAAAGTCAATGGATC |
| 51 | GGGAGTGGTATGTGGCCCT |
| 52 | CCGCTGCCACTGATAGGAA |
| 53 | CAGCAGTAGCGCTATGTCT |
| 54 | AAGGAAGTGGCTTGGAGCA |

TABLE 2
siRNA SEQUENCES for mouse FGF5
(Accession No. NM_010203)

| SEQ ID NO: | SEQUENCE |
|---|---|
| 55 | CCACGAAGCCAGTGTGTTA |
| 56 | AGGGAGAGATTCCAAGAAA |
| 57 | CCTGAACAAGAGAGGGAAA |
| 58 | CCACAGAACTGAAAAGACA |
| 59 | GGAAATATTTGCTGTGTCT |
| 60 | GACTGTAAGTTCAGGGAGA |
| 61 | GGGAGAGATTCCAAGAAAA |
| 62 | GGTACGTGGCCCTGAACAA |
| 63 | GTGCCAAATTTACGGATGA |
| 64 | GTAAGTTCAGGGAGAGATT |
| 65 | AGATCTACCCGGATGGCAA |
| 66 | AGACAGGCCGAGAGTGGTA |
| 67 | TGTAGGAATACGAGGAGTT |
| 68 | CCAAGGAAGCGGCTCGGAA |
| 69 | GAACATAGCAGTTTCCAGT |
| 70 | GCAGAGTGGGCATCGGTTT |
| 71 | TCAGGGGATTGTAGGAATA |
| 72 | ACGGATGACTGTAAGTTCA |
| 73 | GTTCAAGCAGTCCGAGCAA |
| 74 | GAAGTTTCGCTTTGGATGA |
| 75 | GGAAGCGGCTCGGAACATA |
| 76 | AAGCCAGTGTGTTAAGTAT |
| 77 | CATGCAAGTGCCAAATTTA |
| 78 | GATCTACCCGGATGGCAAA |
| 79 | GATTGTAGGAATACGAGGA |
| 80 | CCATGCAAGTGCCAAATTT |
| 81 | GAACTTTCCTTCACCGTCA |
| 82 | GCCAGTGTGTTAAGTATTT |
| 83 | ACAAGAGAGGGAAAGCCAA |
| 84 | CCTAGGAACCCGGGAGACT |
| 85 | AAATATTTGCTGTGTCTCA |
| 86 | CTGTAAGTTCAGGGAGAGA |
| 87 | GGAGAGATTCCAAGAAAAC |
| 88 | CCAAGAAAACAGCTATAAT |
| 89 | GTCCCAGCCCAGTGAAGTA |
| 90 | AGAAGTAGCGCGACGTTTT |
| 91 | AGTCAATGGCTCCCACGAA |
| 92 | GAGGGAAAGCCAAGAGAGG |
| 93 | TGAAGTACAGACTGAAGTT |
| 94 | AGGAAAACTCCATGCAAGT |
| 95 | GTTCAGGGAGAGATTCCAA |
| 96 | AACAAGAGAGGGAAAGCCA |
| 97 | CAAGAGAGGGAAAGCCAAG |
| 98 | GCAGAAGTAGCGCGACGTT |
| 99 | AAGGAAGCGGCTCGGAACA |
| 100 | GCTCGGAACATAGCAGTTT |
| 101 | GTAGGAATACGAGGAGTTT |
| 102 | CAAATTTACGGATGACTGT |
| 103 | GCGATCCACAGAACTGAAA |
| 104 | GAGAGGGAAAGCCAAGAGA |

RNA polymerase III transcribed DNAs contain promoters, such as the U6 promoter. These DNAs can be transcribed to produce small hairpin RNAs in the cell that can function as siRNA or linear RNAs that can function as antisense RNA. The FGF5 modulating compound can contain ribonucleotides, deoxyribonucleotides, synthetic nucleotides, or any suitable combination such that the target RNA and/or gene is inhibited. In addition, these forms of nucleic acid can be single, double, triple, or quadruple stranded. (see for example Bass (2001) Nature, 411:428-429; Elbashir et al., (2001) Nature, 411:494 498; U.S. Pat. No. 6,509,154; and PCT Publication Nos. WO 00/044895, WO 01/036646, WO 99/032619, WO 00/01846, WO 01/029058, WO 00/44914).

A FGF5 modulating compound can be a small molecule that binds to a FGF5 protein and disrupts its function, or conversely, enhances its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate a FGF5 protein can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described below (Werner et al., (2006) Brief Funct. Genomic Proteomic 5(1):32-6).

Knowledge of the primary sequence of a molecule of interest, such as a polypeptide encoded by a FGF5 gene, and the similarity of that sequence with proteins of known function, can provide information as to the inhibitors or antagonists of the protein of interest in addition to agonists. Identification and screening of agonists and antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Test compounds, such as FGF5 modulating compounds, can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76). Numerous means are currently used for random and directed synthesis of saccharide, peptide, and nucleic acid based compounds. Synthetic compound libraries are commercially available from Maybridge Chemical Co. (Trevillet, Cornwall, UK), AMRI (Albany, N.Y.), ChemBridge (San Diego, Calif.), and MicroSource (Gaylordsville, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the presently disclosed subject matter include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. For example, libraries can also include, but are not limited to, peptide-on-plasmid libraries, synthetic small molecule libraries, aptamer libraries, in vitro translation-based libraries, polysome libraries, synthetic peptide libraries, neurotransmitter libraries, and chemical libraries.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/020242, dated Oct. 14, 1993; and Brenner et al., (1992) Proc. Natl. Acad. Sci. USA 89:5381-5383.

Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/108318.

In vitro translation-based libraries include but are not limited to those described in PCT Publication No. WO 91/005058; and Mattheakis et al., (1994) Proc. Natl. Acad. Sci. USA 91:9022-9026.

As used herein, the term “ligand source” can be any compound library described herein, or tissue extract prepared from various organs in an organism's system, that can be used to screen for compounds that would act as an agonist or antagonist of a FGF5 protein. Screening compound libraries listed herein (also see U.S. Pat. No. 7,884,189, which is hereby incorporated by reference in its entirety), in combination with in vivo animal studies, functional and signaling assays described below can be used to identify FGF5 modulating compounds that regulate hair growth or treat hair loss disorders.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815, 5,223,409, and 5,198,346, all to Ladner et al.; Rebar et al., (1993) Science 263:671-673; and PCT Publication No. WO 94/018318.

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT Publication No. WO 95/018972, as well as U.S. Pat. No. 5,712,171 and its corresponding PCT Publication No. WO 96/022529, which are each hereby incorporated by reference in their entireties.

In one non-limiting example, non-peptide libraries, such as a benzodiazepine library (see e.g., Bunin et al., (1994) Proc. Natl. Acad. Sci. USA 91:4708-4712), can be screened. Peptoid libraries, such as that described by Simon et al., (1992) Proc. Natl. Acad. Sci. USA 89:9367-9371, can also be used. Another example of a library that can be used, in which the amide functionalities in peptides have been permethylated to generate a chemically transformed combinatorial library, is described by Ostresh et al. (1994), Proc. Natl. Acad. Sci. USA 91:11138-11142.

Computer modeling and searching technologies permit the identification of compounds, or the improvement of already identified compounds, that can modulate the expression or activity of a FGF5 protein. Having identified such a compound or composition, the active sites or regions of a FGF5 protein can be subsequently identified via examining the sites to which the compounds bind. These sites can be ligand binding sites and can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

The three dimensional geometric structure of a site, for example that of a polypeptide encoded by a FGF5 gene, can be determined by known methods in the art, such as X-ray crystallography, which can determine a complete molecular structure. Solid or liquid phase NMR can be used to determine certain intramolecular distances. Any other experimental method of structure determination can be used to obtain partial or complete geometric structures. The geometric structures can be measured with a complexed ligand, natural or artificial, which can increase the accuracy of the active site structure determined.

Other methods for preparing or identifying peptides that bind to a target are known in the art. Molecular imprinting, for instance, can be used for the de novo construction of macromolecular structures such as peptides that bind to a molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Mosbach, (1994) Trends in Biochem. Sci., 19(9); and Wulff, G., in Polymeric Reagents and Catalysts (Ford, W. T., Ed.) ACS Symposium Series No. 308, pp 186-230, American Chemical Society (1986). One method for preparing mimics of a FGF5 modulating compound involves the steps of: (i) polymerization of functional monomers around a known substrate (the template) that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are prepared by the free radical polymerization of functional monomers, resulting in a compound with a nonbiodegradable backbone. Other methods for designing such molecules include for example drug design based on structure activity relationships, which require the synthesis and evaluation of a number of compounds and molecular modeling.

Screening Assays

FGF5 Modulating Compounds.

A FGF5 modulating compound can be a compound that affects the activity and/or expression of a FGF5 protein in vivo and/or in vitro. FGF5 modulating compounds can be agonists and antagonists of a FGF5 protein, and can be compounds that exert their effect on the activity of a FGF5 protein via the expression, via post-translational modifications, or by other means.

Test compounds or agents which bind to a FGF5 protein, and/or have a stimulatory or inhibitory effect on the activity or the expression of a FGF5 protein, can be identified by two types of assays: (a) cell-based assays which utilize cells expressing a receptor for the FGF5 protein (e.g., FGFR1) or a variant thereof on the cell surface; or (b) cell-free assays, which can make use of isolated FGF5 proteins. In one embodiment, the cells used are DP cells. These assays can employ a biologically active fragment of a FGF5 protein, full-length proteins, or a fusion protein which includes all or a portion of a polypeptide encoded by a FGF5 gene. A FGF5 protein can be obtained from any suitable mammalian species (e.g., human, rat, chick, xenopus, equine, bovine or murine). The assay can be a binding assay comprising direct or indirect measurement of the binding of a test compound. The assay can also be an activity assay comprising direct or indirect measurement of the activity of a FGF5 protein. The assay can also be an expression assay comprising direct or indirect measurement of the expression of FGF5 mRNA nucleic acid sequences or a protein encoded by a FGF5 gene. The various screening assays can be combined with an in vivo assay comprising measuring the effect of the test compound on the symptoms of a hair loss disorder or disease in a subject (for example, androgenetic alopecia, alopecia areata, alopecia totalis, or alopecia universalis), hair growth disorder (for example, hypertrichosis), loss of hair pigmentation in a subject, or even hypotrichosis.

An in vivo assay can also comprise assessing the effect of a test compound on regulating hair growth in known mammalian models that display defective or aberrant hair growth phenotypes or mammals that contain mutations in the open reading frame (ORF) of nucleic acid sequences comprising a FGF5 gene that affects hair growth regulation or hair density, or hair pigmentation. In one embodiment, controlling hair growth can comprise an induction of hair growth or density in the subject. Here, the compound's effect in regulating hair growth can be observed either visually via examining the organism's physical hair growth or loss, or by assessing protein or mRNA expression using methods known in the art.

Assays for screening test compounds that bind to or modulate the activity of a FGF5 protein can also be carried out. The test compound can be obtained by any suitable means, such as from conventional compound libraries. Determining the ability of the test compound to bind to a membrane-bound form of the FGF5 protein can be accomplished via coupling the test compound with a radioisotope or enzymatic label such that binding of the test compound to the cell expressing a FGF5 protein can be measured by detecting the labeled compound in a complex. For example, the test compound can be labeled with ³H, ¹⁴C, ³⁵S, or ¹²⁵I, either directly or indirectly, and the radioisotope can be subsequently detected by direct counting of radioemmission or by scintillation counting. Alternatively, the test compound can be enzymatically labeled with, for example, horseradish peroxidase, alkaline phosphatase, or luciferase, and the enzymatic label detected by determination of conversion of an appropriate substrate to product.

Cell-based assays can comprise contacting a cell expressing a receptor for FGF5 with a test agent and determining the ability of the test agent to modulate (such as increase or decrease) the activity or the expression of the membrane-bound molecule. In one embodiment, the FGF5 receptor is FGFR1 or FGFR2. In another embodiment, the FGFR1 and FGFR2 receptors are isoforms C. Determining the ability of the test agent to modulate the activity of the membrane-bound FGF5 molecule can be accomplished by any method suitable for measuring the activity of such a molecule, such as monitoring downstream signaling events or in vitro cell proliferation (e.g., Rosenquist T A, Martin G R. Dev Dyn. 1996 April; 205(4):379-86; Suzuki et al., J Invest Dermatol. 1998 December; 111(6):963-72, each of which are incorporated by reference in their entireties).

A FGF5 protein or the target of a FGF5 protein can be immobilized to facilitate the separation of complexed from uncomplexed forms of one or both of the proteins. Binding of a test compound to a FGF5 protein or a variant thereof, or interaction of a FGF5 protein with a target molecule in the presence and absence of a test compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix (for example, glutathione-S-transferase (GST) fusion proteins or glutathione-S-transferase fusion proteins can be adsorbed onto glutathione sepharose beads (Sigma Chemical; St. Louis, Mo.) or glutathione derivatized microtiter plates).

A FGF5 protein, or a variant thereof, can also be immobilized via being bound to a solid support. Non-limiting examples of suitable solid supports include glass or plastic slides, tissue culture plates, microtiter wells, tubes, silicon chips, or particles such as beads (including, but not limited to, latex, polystyrene, or glass beads). Any method known in the art can be used to attach a polypeptide (or polynucleotide) corresponding to FGF5 or a variant thereof, or test compound to a solid support, including use of covalent and non-covalent linkages, or passive absorption.

The expression of a FGF5 protein can also be monitored. For example, regulators of the expression of a FGF5 protein can be identified via contacting a cell with a test compound and determining the expression of a protein encoded by a FGF5 gene or FGF5 mRNA nucleic acid sequences in the cell. The expression level of a protein encoded by a FGF5 gene or FGF5 mRNA nucleic acid sequences in the cell in the presence of the test compound is compared to the protein or mRNA expression level in the absence of the test compound. The test compound can then be identified as a regulator of the expression of a FGF5 protein based on this comparison. For example, when expression of a protein encoded by a FGF5 gene or FGF5 mRNA nucleic acid sequences in the cell is statistically or significantly greater in the presence of the test compound than in its absence, the test compound is identified as a stimulator/enhancer of expression of a protein encoded by a FGF5 gene or FGF5 mRNA nucleic acid sequences in the cell. The test compound can be said to be a FGF5 modulating compound (such as an agonist).

Alternatively, when expression of a protein encoded by a FGF5 gene or FGF5 mRNA nucleic acid sequences in the cell is statistically or significantly less in the presence of the test compound than in its absence, the compound is identified as an inhibitor of the expression of a protein encoded by a FGF5 gene or FGF5 mRNA nucleic acid sequences in the cell. The test compound can also be said to be a FGF5 modulating compound (such as an antagonist). The expression level of a protein encoded by a FGF5 gene or FGF5 mRNA nucleic acid sequences in the cell in cells can be determined by methods previously described.

For binding assays, the test compound can be a small molecule which binds to and occupies the binding site of a polypeptide encoded by a FGF5 gene, or a variant thereof. This can make the ligand binding site inaccessible to substrate such that normal biological activity is prevented. Examples of such small molecules include, but are not limited to, small peptides or peptide-like molecules. In binding assays, either the test compound or a polypeptide encoded by a FGF5 gene can comprise a detectable label, such as a fluorescent, radioisotopic, chemiluminescent, or enzymatic label (for example, alkaline phosphatase, horseradish peroxidase, or luciferase). Detection of a test compound which is bound to a polypeptide encoded by a FGF5 gene can then be determined via direct counting of radioemmission, by scintillation counting, or by determining conversion of an appropriate substrate to a detectable product.

Determining the ability of a test compound to bind to a FGF5 protein also can be accomplished using real-time Biamolecular Interaction Analysis (BIA) [McConnell et al., 1992, Science 257, 1906-1912; Sjolander, Urbaniczky, 1991, Anal. Chem. 63, 2338-2345]. BIA is a technology for studying biospecific interactions in real time, without labeling any of the interactants (for example, BIA-Core™). Changes in the optical phenomenon surface plasmon resonance (SPR) can be used as an indication of real-time reactions between biological molecules.

To identify other proteins which bind to or interact with a FGF5 protein and modulate its activity, a polypeptide encoded by a FGF5 gene can be used as a bait protein in a two-hybrid assay or three-hybrid assay (Szabo et al., 1995, Curr. Opin. Struct. Biol. 5, 699-705; U.S. Pat. No. 5,283,317), according to methods practiced in the art. The two-hybrid system is based on the modular nature of most transcription factors, which consist of separable DNA-binding and activation domains.

Functional Assays.

Test compounds can be tested for the ability to increase or decrease the activity of a FGF5 protein, or a variant thereof. Activity can be measured after contacting a purified FGF5 protein, a cell membrane preparation, or an intact cell with a test compound. A test compound that decreases the activity of a FGF5 protein by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for decreasing the activity of a FGF5 protein, for example an antagonist. A test compound that increases the activity of a FGF5 protein by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for increasing the activity of a FGF5 protein, for example an agonist.

Treatment and Prevention

The presently disclosed subject matter also provides a method for treating or preventing a hair-loss disorder in a subject. In one embodiment, the method comprises detecting the presence of an alteration in a FGF5 gene in a sample from the subject, the presence of the alteration being indicative of a hair-loss disorder, or the predisposition to a hair-loss disorder, and, administering to the subject in need a therapeutic treatment against a hair-loss disorder. The therapeutic treatment can be a drug administration (for example, a pharmaceutical composition comprising a siRNA directed to a FGF5 nucleic acid). In one embodiment, the therapeutic molecule to be administered comprises a polypeptide encoded by a FGF5 gene, comprising at least about 75%, at least about 80%, at least about 85%, at least about 90%, at least about 93%, at least about 95%, at least about 97%, at least about 98%, at least about 99%, or 100% of the amino acid sequence of SEQ ID NO: 1 or 3, and exhibits the function of decreasing expression of a protein encoded by a FGF5 gene. This can restore the capacity to initiate hair growth in cells derived from hair follicles or skin. In another embodiment, the therapeutic molecule to be administered comprises a nucleic acid sequence comprising a FGF5 gene that encodes a polypeptide, comprising at least about 75%, at least about 80%, at least about 85%, at least about 90%, at least about 93%, at least about 95%, at least about 97%, at least about 98%, at least about 99%, or 100% of the nucleic acid sequence of SEQ ID NO: 2 or 4, and encodes a polypeptide with the function of decreasing expression of a protein encoded by a FGF5 gene, thus restoring the capacity to initiate hair growth in cells derived from hair follicle cells or skin.

The alteration can be determined at the level of the DNA, RNA, or polypeptide. Optionally, detection can be determined by performing an oligonucleotide ligation assay, a confirmation based assay, a hybridization assay, a sequencing assay, an allele-specific amplification assay, a microsequencing assay, a melting curve analysis, a denaturing high performance liquid chromatography (DHPLC) assay (for example, see Jones et al, (2000) Hum Genet., 106(6):663-8), or a combination thereof. In another embodiment, the detection is performed by sequencing all or part of a FGF5 gene or by selective hybridization or amplification of all or part of a FGF5 gene. A FGF5 gene specific amplification can be carried out before the alteration identification step.

An alteration in a chromosome region occupied by a FGF5 gene can be any form of mutation(s), deletion(s), rearrangement(s) and/or insertions in the coding and/or non-coding region of the locus, alone or in various combination(s). Mutations can include point mutations. Insertions can encompass the addition of one or several residues in a coding or non-coding portion of the gene locus. Insertions can comprise an addition of between 1 and 50 base pairs in the gene locus. Deletions can encompass any region of one, two or more residues in a coding or non-coding portion of the gene locus, such as from two residues up to the entire gene or locus. Deletions can affect smaller regions, such as domains (introns) or repeated sequences or fragments of less than about 50 consecutive base pairs, although larger deletions can occur as well. Rearrangement includes inversion of sequences. The alteration in a chromosome region occupied by a FGF5 gene can result in amino acid substitutions, RNA splicing or processing, product instability, the creation of stop codons, frame-shift mutations, and/or truncated polypeptide production. The alteration can result in the production of a polypeptide encoded by a FGF5 gene with altered function, stability, targeting or structure. The alteration can also cause a reduction, or even an increase in protein expression. In one embodiment, the alteration in the chromosome region occupied by a FGF5 gene can comprise a point mutation, a deletion, or an insertion in a FGF5 gene or corresponding expression product. In another embodiment, the alteration can be a deletion or partial deletion of a FGF5 gene. The alteration can be determined at the level of the DNA, RNA, or polypeptide.

In another embodiment, the method can comprise detecting the presence of altered RNA expression. Altered RNA expression includes the presence of an altered RNA sequence, the presence of an altered RNA splicing or processing, or the presence of an altered quantity of RNA. These can be detected by various techniques known in the art, including sequencing all or part of the RNA or by selective hybridization or selective amplification of all or part of the RNA. In a further embodiment, the method can comprise detecting the presence of altered expression of a polypeptide encoded by a FGF5 gene. Altered polypeptide expression includes the presence of an altered polypeptide sequence, the presence of an altered quantity of polypeptide, or the presence of an altered tissue distribution. These can be detected by various techniques known in the art, including by sequencing and/or binding to specific ligands (such as antibodies).

Various techniques known in the art can be used to detect or quantify altered gene or RNA expression or nucleic acid sequences, which include, but are not limited to, hybridization, sequencing, amplification, and/or binding to specific ligands (such as antibodies). Other suitable methods include allele-specific oligonucleotide (ASO), oligonucleotide ligation, allele-specific amplification, Southern blot (for DNAs), Northern blot (for RNAs), single-stranded conformation analysis (SSCA),), pulsed-field gel electrophoresis (PFGE), isoelectric focusing, fluorescent in situ hybridization (FISH), gel migration, clamped denaturing gel electrophoresis, denaturing HPLC, melting curve analysis, heteroduplex analysis, RNase protection, chemical or enzymatic mismatch cleavage, ELISA, radio-immunoassays (RIA) and immuno-enzymatic assays (IEMA). In one embodiment, the detecting comprises using a northern blot; real time PCR and primers directed to SEQ ID NO: 2 or 4; a ribonuclease protection assay; a hybridization, amplification, or sequencing technique to distinguish SEQ ID NO: 2 or 4; or a combination thereof.

Some of these approaches (such as SSCA and constant gradient gel electrophoresis (CGGE)) are based on a change in electrophoretic mobility of the nucleic acids, as a result of the presence of an altered sequence. According to these techniques, the altered sequence is visualized by a shift in mobility on gels. The fragments can then be sequenced to confirm the alteration. Some other approaches are based on specific hybridization between nucleic acids from the subject and a probe specific for wild type or altered gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand specific for the polypeptide, for example, the use of a specific antibody.

Sequencing.

Sequencing can be carried out using techniques well known in the art, using automatic sequencers. The sequencing can be performed on the complete FGF5 gene or on specific domains thereof, such as those known or suspected to carry deleterious mutations or other alterations.

Amplification.

Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR, allele-specific PCR, or PCR based single-strand conformational polymorphism (SSCP). Amplification usually requires the use of specific nucleic acid primers, to initiate the reaction. Nucleic acid primers useful for amplifying sequences from a FGF5 gene or locus are able to specifically hybridize with a portion of a FGF5 gene locus that flank a target region of the locus, wherein the target region is altered in certain subjects having a hair-loss disorder. In one embodiment, amplification can comprise using forward and reverse PCR primers comprising nucleotide sequences of SEQ ID NO: 2 or 4. Non-limiting amplification methods include, e.g., polymerase chain reaction, PCR (PCR Protocols, A Guide To Methods And Applications, ed. Innis, Academic Press, N. Y., 1990 and PCR Strategies, 1995, ed. Innis, Academic Press, Inc., N.Y.); ligase chain reaction (LCR) (Wu (1989) Genomics 4:560; Landegren (1988) Science 241:1077; Barringer (1990) Gene 89:117); transcription amplification (Kwoh (1989) PNAS 86:1173); and, self-sustained sequence replication (Guatelli (1990) PNAS 87:1874); Q Beta replicase amplification (Smith (1997) J. Clin. Microbiol. 35:1477-1491), automated Q-beta replicase amplification assay (Burg (1996) Mol. Cell. Probes 10:257-271) and other RNA polymerase mediated techniques (e.g., NASBA, Cangene, Mississauga, Ontario; see also Berger (1987) Methods Enzymol. 152:307-316; U.S. Pat. Nos. 4,683,195 and 4,683,202; and Sooknanan (1995) Biotechnology 13:563-564). All the references stated above are incorporated by reference in their entireties.

The presently disclosed subject matter provides for a nucleic acid primer, wherein the primer can be complementary to and hybridize specifically to a portion of a FGF5 coding sequence (e.g., gene or RNA) altered in certain subjects having a hair-loss disorder. Primers of the presently disclosed subject matter can be specific for altered sequences in a FGF5 gene or RNA. By using such primers, the detection of an amplification product indicates the presence of an alteration in a FGF5 gene or the absence of such gene. Primers can also be used to identify single nucleotide polymorphisms (SNPs) located in or around a FGF5 gene locus; SNPs can comprise a single nucleotide change, or a cluster of SNPs in and around a FGF5 gene. Examples of primers of this presently disclosed subject matter can be single-stranded nucleic acid molecules of about 5 to 60 nucleotides in length, or about 8 to about 25 nucleotides in length. The sequence can be derived directly from the sequence of a FGF5 gene. Perfect complementarity is useful to ensure high specificity; however, certain mismatch can be tolerated. For example, a nucleic acid primer or a pair of nucleic acid primers as described above can be used in a method for detecting the presence of or a predisposition to a hair-loss disorder in a subject.

Selective Hybridization.

Hybridization detection methods are based on the formation of specific hybrids between complementary nucleic acid sequences that serve to detect nucleic acid sequence alteration(s). A detection technique involves the use of a nucleic acid probe specific for wild type or altered gene or RNA, followed by the detection of the presence of a hybrid. The probe can be in suspension or immobilized on a substrate or support (for example, as in nucleic acid array or chips technologies). The probe can be labeled to facilitate detection of hybrids. For example, a sample from the subject can be contacted with a nucleic acid probe specific for a wild type FGF5 gene or an altered FGF5 gene, and the formation of a hybrid can be subsequently assessed. In one embodiment, the method comprises contacting simultaneously the sample with a set of probes that are specific, respectively, for a wild type FGF5 gene and for various altered forms thereof. Thus, it is possible to detect directly the presence of various forms of alterations in a FGF5 gene in the sample. Also, various samples from various subjects can be treated in parallel.

According to the presently disclosed subject matter, a probe can be a polynucleotide sequence which is complementary to and can specifically hybridize with a (target portion of a) FGF5 gene or RNA, and that is suitable for detecting polynucleotide polymorphisms associated with alleles of a FGF5 gene (or genes) which predispose to or are associated with a hair-loss disorder. Useful probes are those that are complementary to a FGF5 gene, RNA, or target portion thereof. Probes can comprise single-stranded nucleic acids of between 8 to 1000 nucleotides in length, for instance between 10 and 800, between 15 and 700, or between 20 and 500. Longer probes can be used as well. A useful probe of the presently disclosed subject matter is a single stranded nucleic acid molecule of between 8 to 500 nucleotides in length, which can specifically hybridize to a region of a FGF5 gene or RNA that carries an alteration. For example, the probe can be directed to a chromosome region occupied by a FGF5 gene.

The sequence of the probes can be derived from the sequences of a FGF5 gene and RNA as provided herein. Nucleotide substitutions can be performed, as well as chemical modifications of the probe. Such chemical modifications can be accomplished to increase the stability of hybrids (e.g., intercalating groups) or to label the probe. Some examples of labels include, without limitation, radioactivity, fluorescence, luminescence, and enzymatic labeling.

A guide to the hybridization of nucleic acids is found in e.g., Sambrook, ed., Molecular Cloning: A Laboratory Manual (3^{rd} Ed.), Vols. 1-3, Cold Spring Harbor Laboratory, 2001; Current Protocols In Molecular Biology, Ausubel, ed. John Wiley & Sons, Inc., New York, 2001; Laboratory Techniques In Biochemistry And Molecular Biology: Hybridization With Nucleic Acid Probes, Part I. Theory and Nucleic Acid Preparation, Tijssen, ed. Elsevier, N. Y., 1993.

Specific Ligand Binding. As discussed herein, alteration in a chromosome region occupied by a FGF5 gene or alteration in expression of a FGF5 gene, can also be detected by screening for alteration(s) in a sequence or expression level of a polypeptide encoded by a FGF5 gene. Different types of ligands can be used, such as specific antibodies. In one embodiment, the sample is contacted with an antibody specific for a polypeptide encoded by a FGF5 gene and the formation of an immune complex is subsequently determined. Various methods for detecting an immune complex can be used, such as ELISA, radioimmunoassays (RIA) and immuno-enzymatic assays (IEMA). In one embodiment, levels are measured by ELISA using an antibody directed to SEQ ID NO: 1 or 3; western blot using an antibody directed to SEQ ID NO: 1 or 3; mass spectroscopy, isoelectric focusing, or electrophoresis-based techniques targeting epitopes of SEQ ID NO: 1 or 3; or a combination thereof.

For example, an antibody can be a polyclonal antibody, a monoclonal antibody, as well as fragments or derivatives thereof having substantially the same antigen specificity. Fragments include Fab, Fab′2, or CDR regions. Derivatives include single-chain antibodies, humanized antibodies, or poly-functional antibodies. An antibody specific for a polypeptide encoded by a FGF5 gene can be an antibody that selectively binds such a polypeptide, namely, an antibody raised against a polypeptide encoded by a FGF5 gene or an epitope-containing fragment thereof. Although non-specific binding towards other antigens can occur, binding to the target polypeptide occurs with a higher affinity and can be reliably discriminated from non-specific binding. In one embodiment, the method can comprise contacting a sample from the subject with an antibody specific for a wild type or an altered form of a polypeptide encoded by a FGF5 gene, and determining the presence of an immune complex. Optionally, the sample can be contacted to a support coated with antibody specific for the wild type or altered form of a polypeptide encoded by a FGF5 gene. In one embodiment, the sample can be contacted simultaneously, or in parallel, or sequentially, with various antibodies specific for different forms of a polypeptide encoded by a FGF5 gene, such as a wild type and various altered forms thereof.

Gene Therapy and Protein Replacement Methods

Delivery of nucleic acids into viable cells can be effected ex vivo, in situ, or in vivo by use of vectors, such as viral vectors (e.g., lentivirus, adenovirus, adeno-associated virus, or a retrovirus), or ex vivo by use of physical DNA transfer methods (e.g., liposomes or chemical treatments). Non-limiting techniques suitable for the transfer of nucleic acid into mammalian cells in vitro include the use of liposomes, electroporation, microinjection, cell fusion, DEAE-dextran, and the calcium phosphate precipitation method (See, for example, Anderson, (1998) Nature, 392(6679):25 (1998)). Introduction of a nucleic acid or a gene encoding a polypeptide of the presently disclosed subject matter can also be accomplished with extrachromosomal substrates (transient expression) or artificial chromosomes (stable expression). Cells can also be cultured ex vivo in the presence of therapeutic compositions of the presently disclosed subject matter in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., (1992) J Gen Virol. 73(Pt 6):1533-6), adenovirus (Berkner (1992) Curr Top Microbiol Immunol. 158:39-66; Berkner (1988) Biotechniques, 6(7):616-29; Gorziglia and Kapikian (1992) J Virol. 66(7):4407-12; Quantin et al., (1992) Proc Nall Acad Sci USA. 89(7):2581-4; Rosenfeld et al., (1992) Cell. 68(1):143-55; Wilkinson et al., (1992) Nucleic Acids Res. 20(9):2233-9; Stratford-Perricaudet et al., (1990) Hum Gene Ther. 1(3):241-56), vaccinia virus (Moss (1992) Curr Opin Biotechnol. 3(5):518-22), adeno-associated virus (Muzyczka, (1992) Curr Top Microbiol Immunol. 158:97-129; Ohi et al., (1990) Gene. 89(2):279-82), herpesviruses including HSV and EBV (Margolskee (1992) Curr Top Microbiol Immunol. 158:67-95; Johnson et al., (1992) Brain Res Mol Brain Res. 12(1-3):95-102; Fink et al., (1992) Hum Gene Ther. 3(1):11-9; Breakefield and Geller (1987) Mol Neurobiol. 1(4):339-71; Freese et al., (1990) Biochem Pharmacol. 40(10):2189-99), and retroviruses of avian (Bandyopadhyay and Temin (1984) Mol Cell Biol. 4(4):749-54; Petropoulos et al., (1992) J Virol. 66(6):3391-7), murine (Miller et al. (1992) Mol Cell Biol. 12(7):3262-72; Miller et al., (1985) J Virol. 55(3):521-6; Sorge et al., (1984) Mol Cell Biol. 4(9):1730-7; Mann and Baltimore (1985) J Virol. 54(2):401-7; Miller et al., (1988) J Virol. 62(11):4337-45), and human origin (Shimada et al., (1991) J Clin Invest. 88(3):1043-7; Helseth et al., (1990) J Virol. 64(12):6314-8; Page et al., (1990) J Virol. 64(11):5270-6; Buchschacher and Panganiban (1992) J Virol. 66(5):2731-9).

Non-limiting examples of in vivo gene transfer techniques include transfection with viral (e.g., retroviral) vectors (see U.S. Pat. No. 5,252,479, which is incorporated by reference in its entirety) and viral coat protein-liposome mediated transfection (Dzau et al., (1993) Trends in Biotechnology 11:205-210), incorporated entirely by reference). For example, naked DNA vaccines are generally known in the art; see Brower, (1998) Nature Biotechnology, 16:1304-1305, which is incorporated by reference in its entirety. Gene therapy vectors can be delivered to a subject by, for example, intravenous injection, local administration (see, e.g., U.S. Pat. No. 5,328,470) or by stereotactic injection (see, e.g., Chen, et al., (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of gene therapy protocols and methods see Anderson et al. (1992) Science 256:808-813; U.S. Pat. Nos. 5,252,479, 5,747,469, 6,017,524, 6,143,290, 6,410,010 6,511,847; and U.S. Application Publication Nos. 2002/0077313 and 2002/00069, which are all hereby incorporated by reference in their entireties. For additional reviews of gene therapy technology, see Friedmann (1989) Science, 244:1275-1281; Verma, Scientific American: 68-84 (1990); Miller (1992) Nature, 357: 455-460; Kikuchi et al. (2008) J Dermatol Sci. 50(2):87-98; Isaka et al. (2007) Expert Opin Drug Deliv. 4(5):561-71; Jager et al. (2007) Curr Gene Ther. 7(4):272-83; Waehler et al. (2007) Nat Rev Genet. 8(8):573-87; Jensen et al. (2007) Ann Med. 39(2):108-15; Herweijer et al. (2007) Gene Ther. 14(2):99-107; Eliyahu et al. (2005) Molecules 10(1):34-64; and Altaras et al. (2005) Adv Biochem Eng Biotechnol. 99:193-260, all of which are hereby incorporated by reference in their entireties.

Protein replacement therapy can increase the amount of protein by exogenously introducing wild-type or biologically functional protein by way of infusion. A replacement polypeptide can be synthesized according to known chemical techniques or can be produced and purified via known molecular biological techniques. Protein replacement therapy has been developed for various disorders. For example, a wild-type protein can be purified from a recombinant cellular expression system (e.g., mammalian cells or insect cells-see U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al. and U.S. Pat. No. 5,879,680 to Ginns et al.), human placenta, or animal milk (see U.S. Pat. No. 6,188,045 to Reuser et al.), or other sources known in the art. After the infusion, the exogenous protein can be taken up by tissues through non-specific or receptor-mediated mechanism.

A polypeptide encoded by a FGF5 gene can also be delivered in a controlled release system. For example, the polypeptide can be administered using intravenous infusion, an implantable osmotic pump, a transdermal patch, liposomes, or other modes of administration. In one embodiment, a pump can be used (see Sefton (1987) Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target thus requiring only a fraction of the systemic dose (see, e.g., Goodson, in Medical Applications of Controlled Release, supra, vol. 2, pp. 115-138 (1984)). Other controlled release systems are discussed in the review by Langer (Science (1990) 249:1527-1533).

Pharmaceutical Compositions and Administration for Therapy

FGF5 proteins and FGF5 modulating compounds of the presently disclosed subject matter can be administered to the subject once (e.g., as a single injection or deposition). Alternatively, FGF5 proteins and FGF5 modulating compounds can be administered once or twice daily to a subject in need thereof for a period of from about two to about twenty-eight days, or from about seven to about ten days. FGF5 proteins and FGF5 modulating compounds can also be administered once or twice daily to a subject for a period of 1, 2, 3, 4, 5, 6, 7, 8, 9, 10, 11, 12 times per year, or a combination thereof. Furthermore, FGF5 proteins and FGF5 modulating compounds of the presently disclosed subject matter can be co-administrated with another therapeutic. Where a dosage regimen comprises multiple administrations, the effective amount of the FGF5 proteins and FGF5 modulating compounds administered to the subject can comprise the total amount of gene product administered over the entire dosage regimen.

FGF5 proteins and FGF5 modulating compounds can be administered to a subject by any means suitable for delivering the FGF5 proteins and FGF5 modulating compounds to cells of the subject, such as the dermis, epidermis, dermal papilla cells, or hair follicle cells. For example, FGF5 proteins and FGF5 modulating compounds can be administered by methods suitable to transfect cells. Transfection methods for eukaryotic cells are well known in the art, and include direct injection of the nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

The compositions of this presently disclosed subject matter can be formulated and administered to reduce the symptoms associated with a hair-loss disorder by any means that produces contact of the active ingredient with the agent's site of action in the body of a subject, such as a human or animal (e.g., a dog, cat, or horse). They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

A therapeutically effective dose of FGF5 modulating compounds can depend upon a number of factors known to those or ordinary skill in the art. The dose(s) of the FGF5 modulating compounds can vary, for example, depending upon the identity, size, and condition of the subject or sample being treated, further depending upon the route by which the composition is to be administered, if applicable, and the effect which the practitioner desires the FGF5 modulating compounds to have upon the nucleic acid or polypeptide of the presently disclosed subject matter. These amounts can be readily determined by a skilled artisan. Any of the therapeutic applications described herein can be applied to any subject in need of such therapy, including, for example, a mammal such as a dog, a cat, a cow, a horse, a rabbit, a monkey, a pig, a sheep, a goat, or a human.

Pharmaceutical compositions for use in accordance with the presently disclosed subject matter can be formulated in conventional manner using one or more physiologically acceptable carriers or excipients. The therapeutic compositions of the presently disclosed subject matter can be formulated for a variety of routes of administration, including systemic and topical or localized administration. Techniques and formulations generally can be found in Remington's The Science and Practice of Pharmacy, 20^{th} ed. Lippincott Williams & Wilkins, Philadelphia, Pa. (2000), the entire disclosure of which is herein incorporated by reference. For systemic administration, an injection is useful, including intramuscular, intravenous, intraperitoneal, and subcutaneous. For injection, the therapeutic compositions of the presently disclosed subject matter can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the presently disclosed subject matter are characterized as being at least sterile and pyrogen-free. These pharmaceutical formulations include formulations for human and veterinary use.

According to the presently disclosed subject matter, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

The presently disclosed subject matter also provides for a kit that comprises a pharmaceutically acceptable carrier and a FGF5 modulating compound identified using the screening assays of the presently disclosed subject matter packaged with instructions for use. For modulators that are antagonists of the activity of a FGF5 protein, or which reduce the expression of a FGF5 protein, the instructions would specify use of the pharmaceutical composition for promoting the loss of hair on the body surface of a mammal (for example, arms, legs, bikini area, face).

For FGF5 modulating compounds that are agonists of the activity of a FGF5 protein or increase the expression of one or more proteins encoded by the FGF5 gene, the instructions would specify use of the pharmaceutical composition for regulating hair growth. In one embodiment, the instructions would specify use of the pharmaceutical composition for the treatment of hair loss disorders. In a further embodiment, the instructions would specify use of the pharmaceutical composition for restoring hair pigmentation. For example, administering an agonist can reduce hair graying in a subject.

A pharmaceutical composition containing a FGF5 modulating compound can be administered in conjunction with a pharmaceutically acceptable carrier, for any of the therapeutic effects discussed herein. Such pharmaceutical compositions can comprise, for example antibodies directed to polypeptides encoded by genes comprising a FGF5 gene, or variants thereof, or agonists and antagonists of a polypeptide encoded by a FGF5 gene. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

Sterile injectable solutions can be prepared by incorporating the FGF5 modulating compound (e.g., a polypeptide or antibody) in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

In some embodiments, the FGF5 modulating compound can be applied via transdermal delivery systems, which slowly releases the active compound for percutaneous absorption. Permeation enhancers can be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Various routes of administration and various sites of cell implantation can be utilized, such as, subcutaneous or intramuscular, in order to introduce the aggregated population of cells into a site of preference. Once implanted in a subject (such as a mouse, rat, or human), the aggregated cells can then stimulate the formation of a hair follicle and the subsequent growth of a hair structure at the site of introduction. In another embodiment, transfected cells (for example, cells expressing a protein encoded by a FGF5 gene are implanted in a subject to promote the formation of hair follicles within the subject. In further embodiments, the transfected cells are cells derived from the end bulb of a hair follicle (such as dermal papilla cells or dermal sheath cells). Aggregated cells (for example, cells grown in a hanging drop culture) or transfected cells (for example, cells produced as described herein) maintained for 1 or more passages can be introduced (or implanted) into a subject (such as a rat, mouse, dog, cat, human, and the like).

“Subcutaneous” administration can refer to administration just beneath the skin (i.e., beneath the dermis). Generally, the subcutaneous tissue is a layer of fat and connective tissue that houses larger blood vessels and nerves. The size of this layer varies throughout the body and from person to person. The interface between the subcutaneous and muscle layers can be encompassed by subcutaneous administration.

This mode of administration can be feasible where the subcutaneous layer is sufficiently thin so that the factors present in the compositions can migrate or diffuse from the locus of administration and contact the hair follicle cells responsible for hair formation. Thus, where intradermal administration is utilized, the bolus of composition administered is localized proximate to the subcutaneous layer.

Administration of the cell aggregates (such as DP or DS aggregates) is not restricted to a single route, but can encompass administration by multiple routes. For instance, exemplary administrations by multiple routes include, among others, a combination of intradermal and intramuscular administration, or intradermal and subcutaneous administration. Multiple administrations can be sequential or concurrent. Other modes of application by multiple routes will be apparent to the skilled artisan.

In other embodiments, this implantation method will be a one-time treatment for some subjects. In further embodiments of the presently disclosed subject matter, multiple cell therapy implantations will be required. In some embodiments, the cells used for implantation will generally be subject-specific genetically engineered cells. In another embodiment, cells obtained from a different species or another individual of the same species can be used. Thus, using such cells can require administering an immunosuppressant to prevent rejection of the implanted cells. Such methods have also been described in U.S. Pat. No. 7,419,661 and PCT application publication WO 2001/32840, and are hereby incorporated by reference.

Inhibitors

The inhibitors can comprise peptides (such as antibodies or fragments thereof), small molecules, nucleic acids (such as siRNA or antisense RNA), or other agents) that can bind to a polypeptide molecule encoded by a gene of interest and/or molecules that have an inhibitory effect on the biological activity of a protein of interest or its expression.

As used herein, a “FGF5 inhibitor” refers to a compound that interacts with a FGF5 gene or a FGF5 protein or polypeptide and inhibits its activity and/or its expression. The compound can decrease the activity or expression of a protein encoded by FGF5.

In one embodiment, a FGF5 inhibitor can be a peptide fragment that binds a protein comprising SEQ ID NO: 1 or 3. For example, the fragment can encompass any portion of at least about 8 consecutive amino acids of SEQ ID NO: 1 or 3. The fragment can comprise at least about 10 consecutive amino acids, at least about 20 consecutive amino acids, at least about 30 consecutive amino acids, at least about 40 consecutive amino acids, at least about 50 consecutive amino acids, at least about 60 consecutive amino acids, or at least about 75 consecutive amino acids of SEQ ID NO: 1 or 3. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England).

An inhibitor of the presently disclosed subject matter can be a protein, such as an antibody (monoclonal, polyclonal, humanized, chimeric, or fully human), or a binding fragment thereof, directed against a polypeptide encoded by SEQ ID NO: 1 or 3. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

An inhibitor of the presently disclosed subject matter can also be a small molecule that binds to a protein and disrupts its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate a protein can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries (Werner et al., (2006) Brief Funct. Genomic Proteomic 5(1):32-6). In some embodiments, the agent is a small molecule that binds, interacts, or associates with a target protein or RNA. Such a small molecule can be an organic molecule that, when the target is an intracellular target, is capable of penetrating the lipid bilayer of a cell to interact with the target. Small molecules include, but are not limited to, toxins, chelating agents, metals, and metalloid compounds. Small molecules can be attached or conjugated to a targeting agent so as to specifically guide the small molecule to a particular cell.

Pharmaceutical Compositions and Administration for Therapy

An inhibitor or agonist of the presently disclosed subject matter can be incorporated into pharmaceutical compositions suitable for administration, for example the inhibitor and a pharmaceutically acceptable carrier.

According to the presently disclosed subject matter, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition of the presently disclosed subject matter can be administered in conjunction with a pharmaceutically acceptable carrier, for any of the therapeutic effects discussed herein. Such pharmaceutical compositions can comprise, for example antibodies directed to polypeptides. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

A pharmaceutical composition of the presently disclosed subject matter is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the inhibitor (e.g., a polypeptide or antibody or small molecule) or agonist of the presently disclosed subject matter in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier and subsequently swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The dosage administered can be a therapeutically effective amount of the composition sufficient to result in amelioration of symptoms of alopecia areata, and can vary depending upon known factors such as the pharmacodynamic characteristics of the active ingredient and its mode and route of administration; age, sex, health and weight of the recipient; nature and extent of symptoms; kind of concurrent treatment, frequency of treatment and the effect desired.

In some embodiments, the effective amount of the administered FGF5 modulating compound (e.g., a compound described herein that is directed to FGF5) is at least about 0.0001 μg/kg body weight, at least about 0.00025 μg/kg body weight, at least about 0.0005 μg/kg body weight, at least about 0.00075 μg/kg body weight, at least about 0.001 μg/kg body weight, at least about 0.0025 μg/kg body weight, at least about 0.005 μg/kg body weight, at least about 0.0075 μg/kg body weight, at least about 0.01 μg/kg body weight, at least about 0.025 μg/kg body weight, at least about 0.05 μg/kg body weight, at least about 0.075 μg/kg body weight, at least about 0.1 μg/kg body weight, at least about 0.25 μg/kg body weight, at least about 0.5 μg/kg body weight, at least about 0.75 μg/kg body weight, at least about 1 μg/kg body weight, at least about 5 μg/kg body weight, at least about 10 μg/kg body weight, at least about 25 μg/kg body weight, at least about 50 μg/kg body weight, at least about 75 μg/kg body weight, at least about 100 μg/kg body weight, at least about 150 μg/kg body weight, at least about 200 μg/kg body weight, at least about 250 μg/kg body weight, at least about 300 μg/kg body weight, at least about 350 μg/kg body weight, at least about 400 μg/kg body weight, at least about 450 μg/kg body weight, at least about 500 μg/kg body weight, at least about 550 μg/kg body weight, at least about 600 μg/kg body weight, at least about 650 μg/kg body weight, at least about 700 μg/kg body weight, at least about 750 μg/kg body weight, at least about 800 μg/kg body weight, at least about 850 μg/kg body weight, at least about 900 μg/kg body weight, at least about 950 μg/kg body weight, at least about 1000 μg/kg body weight, at least about 2000 μg/kg body weight, at least about 3000 μg/kg body weight, at least about 4000 μg/kg body weight, at least about 5000 μg/kg body weight, at least about 6000 μg/kg body weight, at least about 7000 μg/kg body weight, at least about 8000 μg/kg body weight, at least about 95000 μg/kg body weight, or at least about 10,000 μg/kg body weight.

Toxicity and therapeutic efficacy of therapeutic compositions of the presently disclosed subject matter can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutic agents that exhibit large therapeutic indices are useful. Therapeutic compositions that exhibit some toxic side effects can be used.

Unless otherwise defined, all technical and scientific terms used herein have the same meaning as commonly understood by one of ordinary skill in the art to which this presently disclosed subject matter belongs. Exemplary methods and materials are described below, although methods and materials similar or equivalent to those described herein can also be used in the practice or testing of the presently disclosed subject matter.

Those skilled in the art will recognize, or be able to ascertain, using no more than routine experimentation, numerous equivalents to the specific substances and procedures described herein. Such equivalents are considered to be within the scope of this presently disclosed subject matter, and are covered by the following claims.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the presently disclosed subject matter. The following examples illustrate the exemplary modes of making and practicing the presently disclosed subject matter. However, the scope of the presently disclosed subject matter is not limited to specific embodiments disclosed in these Examples, which are for purposes of illustration only, since alternative methods can be utilized to obtain similar results.

### Example 1

### Mutation in FGF5 Underlies Trichomegaly with Hypertrichosis

Hair follicles are a unique mammalian organ as they have the ability to continuously regenerate, and throughout their lifetime go through cycles of growth (anagen), regression (catagen) and rest (telogen). In humans, different body sites contain follicles with varying anagen:telogen ratios, for example, hair on the scalp has a long anagen duration (years), then just a short telogen (3 months), enabling growth of long hair. In contrast, hair on the upper arm has an anagen of approximately 28 days (in Caucasian males), followed by a comparatively long telogen of 80 days (P1) resulting in short hair growth at this site An interesting phenotype has been observed in angora mice, which have abnormally long coat hair due to a prolonging of the anagen phase, and a disrupted anagen:telogen ratio (P2). The angora phenotype has been described for many years as a recessive mutation in the mouse, and in 1994, the causative mutation was first found to reside within the fibroblast growth factor 5 (FGF5) gene (P3). Subsequently, FGF5mutations have been described in cats, dogs, goats and rabbits all with the angora phenotype, however, a human counterpart has not yet been identified.

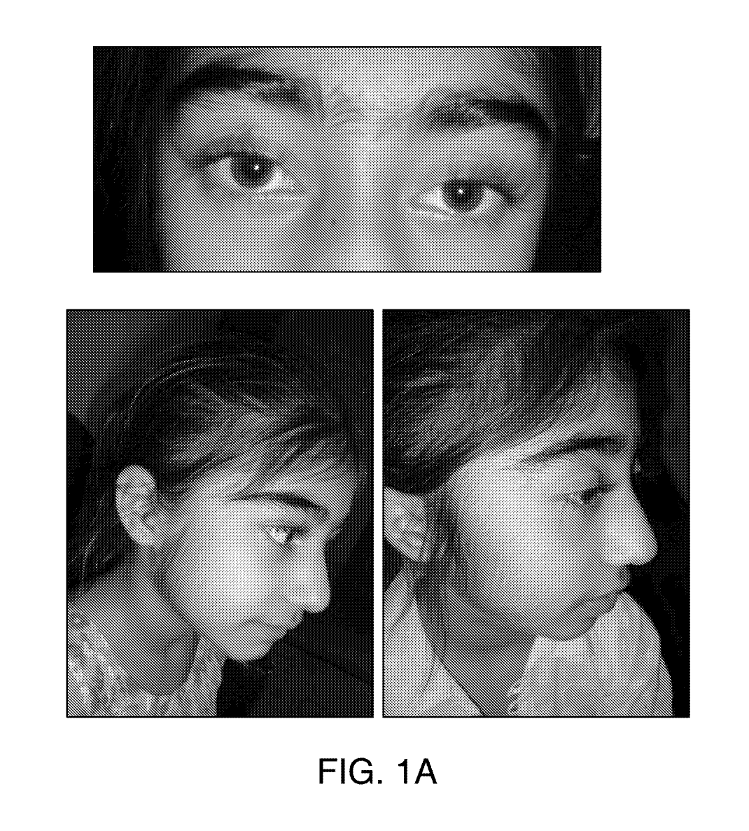

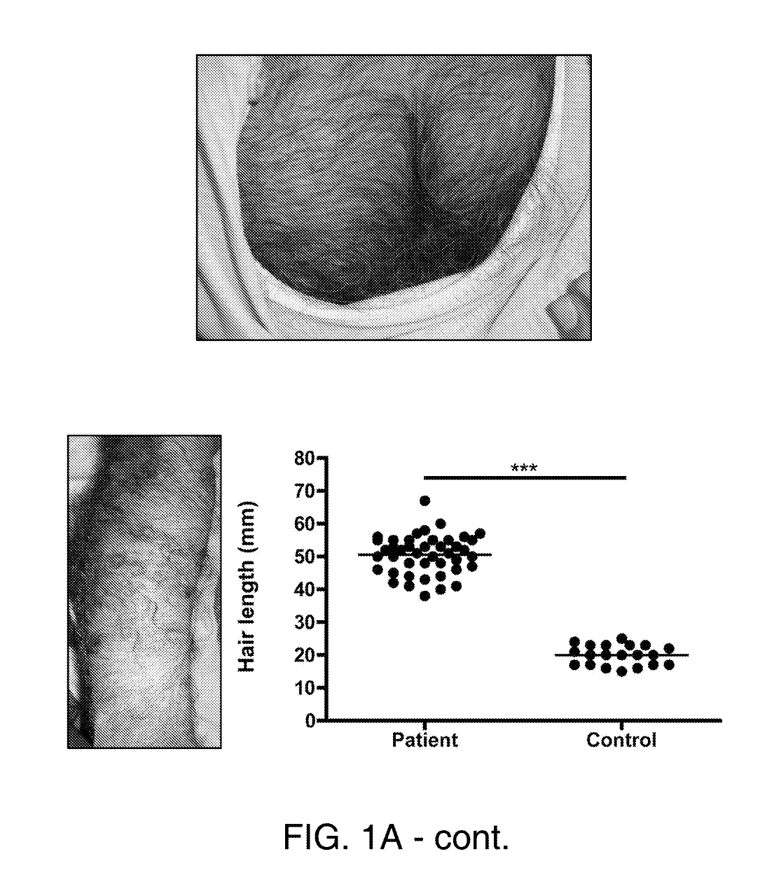

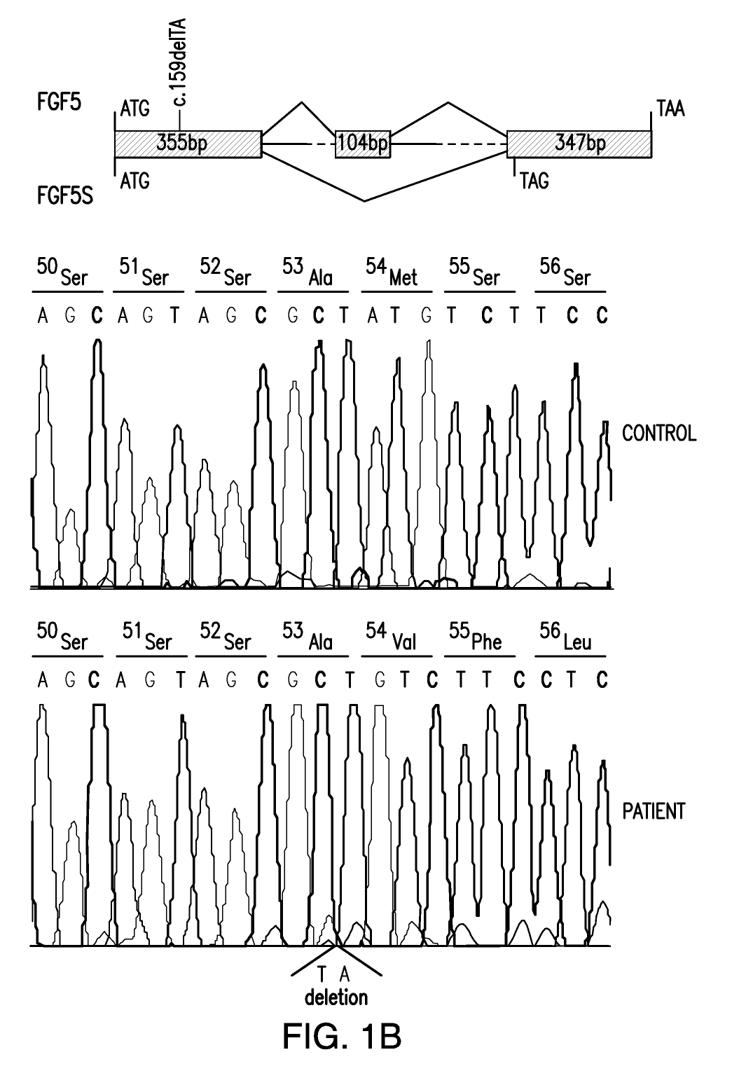

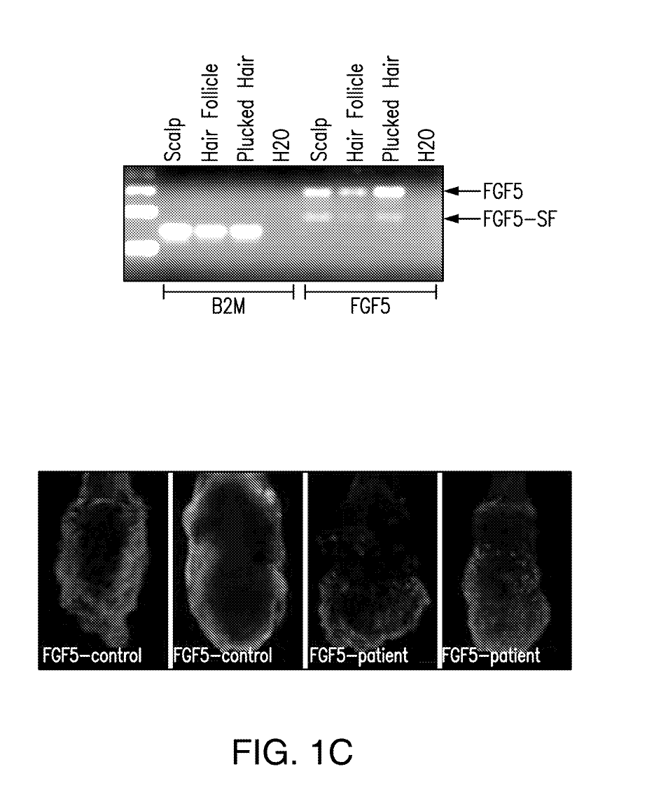

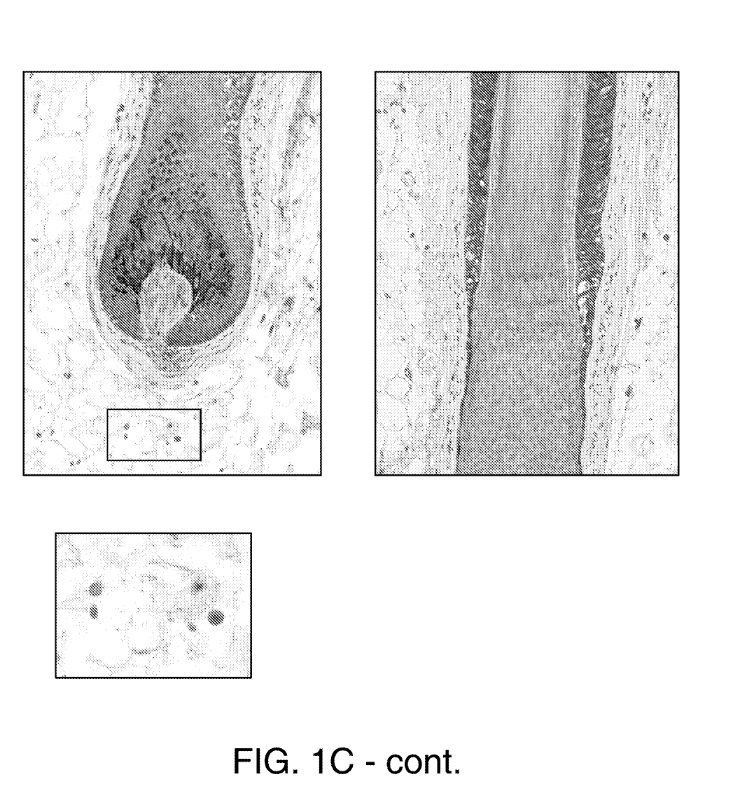

The inventors describe in this example the equivalent of the angora phenotype in humans, in a small consanguineous family from Pakistan who presented with trichomegaly with congenital hypertrichosis in an autosomal recessive inheritance pattern. In addition to trichomegaly, the hypertrichosis observed in this family was also evident on the face and arms, in hairs that are usually in telogen, and was not due to a vellus to terminal switch, or an increased number of hair follicles (FIG. 1A). Hair fibers collected from the forearm of an affected male were 2.5× longer than unaffected males indicating an increased anagen duration in patients (FIG. 1A).

Whole exome sequencing was used to determine the causative mutation and identified a 2 bp deletion in exon 1 of the FGF5 gene (159delTA) that is not a SNP, and not present in 50 controls (FIG. 1B). This deletion results in an out-of-frame transcript with different consequences in the two different splice forms of FGF5. Alternative splicing results in two variants of FGF5, one producing a 268 amino acid protein (FGF5), and a second, shorter 123 amino acid protein (FGF5S) (P4). The TA deletion observed within this family causes a frameshift from amino acid 53, resulting in a premature stop codon in FGF5 most likely resulting in nonsense mediated mRNA decay and loss of function. In FGF5S, the frameshift results in a delayed termination codon and it is unlikely that the mutant protein is functional.

PCR was used to amplify fragments of the two variants, and found both were expressed within scalp skin, but also within plucked hair fibers from the forearms of affected individuals that contain remnants of follicular outer root sheath. To elucidate if FGF5 was present within outer root sheath of patient follicles, whole mount immunofluorescence was performed on hairs plucked from patient forearms, and controls with antibodies specific for both forms of the protein. While FGF5 and FGF5S were both present in the tissue surrounding plucked fibers from unaffected individuals, they were absent from fibers obtained from patients (FIG. 1C). Immunohistochemistry on normal scalp skin also confirmed the presence of FGF5 and FGF5S within the outer root sheath of hair follicles, but interestingly, expression is also observed in small round macrophage cells that surround the follicle.

Within mouse skin, it was proposed that FGF5 secreted by macrophages stimulated cells within the dermal papilla, located at the base of the follicle, to initiate exit from anagen, and entry into catagen (P5). If a similar mechanism is operational in humans, then a mutation in FGF5 explains the prolonged anagen phenotype, as the stimulus for catagen entry has been lost. It also implies to regulation of the follicle cycle by immune cells within the follicular macro environment. Even without FGF5, hair follicles in these patients eventually enter into regression, and then move through to telogen as evidenced by the presence of telogen hairs within our plucked hair samples. This highlights that, either there are other stimuli for catagen that eventually compensate for the absence of FGF5.

Interestingly, in a study of dogs coats in 2009, variation of the coat was attributed to three genes, with mutation in FGF5 regulating hair length across breeds (P6). This study is the first of its kind to utilize whole exome sequencing to study a human hair disorder, and mutations in FGF5 underlie the anagen:telogen ratio of follicles, and subsequently their hair length. Since this phenotype is most visible in the facial hair and eyelashes, it raises the possibility of therapeutic targeting of FGF5 to enhance eyelash growth. Moreover, we cannot exclude the possibility that FGF5 is operating in scalp follicles as well, and therefore, inhibition of FGF5 in scalp hair may also provide therapeutic benefit for patients with short anagen.

### REFERENCES

P1. S. V. Seago, F. J. Ebling, Br J Dermatol 113, 9 (July, 1985).
P2. P. R. Pennycuik, K. A. Raphael, Genet Res 44, 283 (December, 1984).
P3. J. M. Hebert, T. Rosenquist, J. Gotz, G. R. Martin, Cell 78, 1017 (Sep. 23, 1994).
P4. K. Ozawa et al., J Biol Chem 273, 29262 (Oct. 30, 1998).
P5. S. Suzuki, Y. Ota, K. Ozawa, T. Imamura, J Invest Dermatol 114, 456 (March, 2000).
P6. R. Langebaek, Nord Vet Med 38, 383 (November-December, 1986).

What is claimed:

1. A method for inducing hair growth in a subject in need thereof, comprising: administering to the subject an effective amount of an inhibitor of FGF5, thereby inducing hair growth in the subject.

2. The method of claim 1, wherein the inhibitor comprises an antibody that specifically binds to a protein comprising SEQ ID NO: 1 or 3.

3. The method of claim 1, wherein the subject is afflicted with a hair-loss disorder.

4. The method of claim 3, wherein the hair-loss disorder comprises thinning eyelashes, androgenetic alopecia, telogen effluvium, alopecia areata, telogen effluvium, tinea capitis, alopecia totalis, hypotrichosis, hereditary hypotrichosis simplex, or alopecia universalis.

5. The method of claim 3, further comprising determining whether the inhibitor administered induced hair growth in the subject afflicted with a hair loss disorder as compared to the subject's hair growth prior to treatment with the inhibitor.

6. The method of claim 1, wherein the inhibitor is an antisense RNA that specifically inhibits expression of the gene that encodes the FGF5 protein; a siRNA that specifically targets the gene that encodes the FGF5 protein; or a small molecule.

7. The method of claim 6, wherein the siRNA is directed to a human nucleic acid sequence comprising SEQ ID NO: 2 or 4.

8. The method of claim 6, wherein the antisense RNA specifically binds to a human nucleic acid sequence comprising SEQ ID NO: 2 or 4.

9. A method for reducing hair growth in a subject, comprising administering to the subject an effective amount of an activator of FGF5, thereby reducing hair growth in the subject.

10. The method of claim 9, wherein the subject is afflicted with a hair-growth disorder.

11. The method of claim 10, wherein the hair-growth disorder comprises X-linked hypertrichosis, generalized hypertrichosis terminalis with gingival hyperplasia, generalized hypertrichosis terminalis, autosomal recessive hypertrichosis, Cantu syndrome, Ambras type hypertrichosis, or trichomegaly.

12. The method of claim 10, further comprising determining whether the activator administered reduced hair growth in the subject afflicted with a hair-growth disorder as compared to the subject's hair growth prior to treatment with the activator.

13. The method of claim 9, wherein the activator is a polypeptide comprising SEQ ID NO: 1 or 3, or a fragment thereof; or a peptidomimetic comprising SEQ ID NO: 1 or 3.

14. A method for detecting the presence of or a predisposition to a hair-loss disorder in a human subject, comprising:
(a) obtaining a biological sample from a human subject; and
(b) detecting whether or not there is an alteration in the expression of FGF5 in the subject as compared to a subject not afflicted with a hair-loss disorder.

15. The method of claim 14, wherein the hair-loss disorder comprises thinning eyelashes, androgenetic alopecia, telogen effluvium, alopecia areata, telogen effluvium, tinea capitis, alopecia totalis, hypotrichosis, hereditary hypotrichosis simplex, or alopecia universalis.

16. The method of claim 14, wherein the detecting comprises detecting whether there is an alteration in the FGF5 gene locus.

17. The method of claim 16, wherein the alteration comprises a 2 bp deletion in exon 1 of the FGF5 gene.

18. The method of claim 17, wherein the deletion comprises a thymine and adenine at positions 159 and 160.

19. A diagnostic kit for determining whether a sample from a subject exhibits reduced FGF5 expression or exhibits an FGF5 gene mutation, comprising nucleic acid primers that specifically hybridize to and prime a polymerase reaction from FGF5.

20. The kit of claim 19, wherein the primers are directed to SEQ ID NOS: 2 or 4.
